(19) United States
(12) Patent Application Publication
(10) Pub. No.: US 2024/0260641 A1
(43) Pub. Date: Aug. 8, 2024

(54) EXTRUDED HASHISH PRODUCT AND INDUSTRIAL PROCESS FOR MAKING SAME

(71) Applicant: HEXO OPERATIONS INC., GATINEAU (CA)

(72) Inventor: Jamie Savard, Gatineau (CA)

(21) Appl. No.: 18/290,206

(22) PCT Filed: May 12, 2022
(86) PCT No.: PCT/CA2022/050757

§ 371 Date: Nov. 10, 2023

Related U.S. Application Data
(60) Provisional application No. 63/187,760, filed on May 12, 2021.

(51) Int. Cl. A24B 15/16; A24B 15/18

(52) U.S. Cl. CPC A24B 15/16; A24B 15/18

(57) ABSTRACT

The present disclosure relates to hashish products and processes for manufacturing same. The processes may comprise mixing pre-treated isolated cannabis trichomes under conditions sufficient to obtain a resinous mixture and retrieving at least a portion of the resinous mixture through an extrusion die. The hashish product may comprise a not negligible content in one or more acid form of one or more cannabinoid(s).

### CROSS-REFERENCE TO RELATED APPLICATION

This application claims priority from U.S. Provisional Patent Application 63/187,760 filed on May 12, 2021 and incorporated by reference herein.

### TECHNICAL FIELD

This application generally relates to the field of extruded hashish products as well as industrial methods of manufacturing same.

### BACKGROUND

With stage-wise legalization of cannabis-based consumer products in Canada and eventually in various other areas in the world, advancements in extraction technology, industrial scale production and accessibility to a wide variety of forms have accelerated to fulfill emerging demands. Hashish (or hash) is one example of a cannabis-based product, typically used for recreational or medicinal (i.e., health and wellness) purposes, for which there is an increasing consumer demand.

Hashish is a concentrated derivative of cannabis plants, which is extracted from stalked resin glands known as trichomes. It contains the same active ingredients as marijuana—including tetrahydrocannabinol (“THC”) and other cannabinoids—yet at higher concentration levels than the un-sifted buds or leaves from which marijuana is made, which is tantamount to higher potency. The trichomes are usually collected (isolated from the cannabis plant material) by hand, by mechanical beating of the plants or by submersing the cannabis plants in icy water and then using small sieves to isolate the trichomes. Alternatively, mechanical separation may be used to isolate trichomes from the plant, such as sieving through a screen by hand or in motorized tumblers, as described for example in WO 2019/161509. Isolated trichomes have a powder appearance which is mostly comprised of the bulbous, crystal formations on the tip of the glands and are typically referred to as “kief”.

Traditionally, hashish is obtained by pressing kief manually. In an industrial setting, however, manual pressing is hardly scalable and affords poor yield—instead, hashish is obtained by pressing kief in a mechanical press.

Using a mechanical press to press the isolated trichomes into a mold produces a cohesive mass from the isolated trichomes (i.e., hashish product) in the form afforded by the mold. For example, the isolated trichomes can be pressed in a mold affording the shape of individual “bricks”. During pressing, heat may be applied to the isolated trichomes via the pressing plates to cause a release of resin from the trichomes and decarboxylate the cannabinoids (activate the acid form of the cannabinoids). Alternatively, heat may be applied to the pressed trichomes after the pressing step for substantially the same purposes and then, typically, hashish manufacturers will perform a second pressing step after such heating to further ensure cohesiveness of the hashish product. From a production perspective, this batch-like approach to manufacturing hash (e.g., applying heat on a per hashish unit basis after or during pressing) can be labor intensive, reduce volume throughput (in terms of hashish units produced) and negatively affect overall efficiency of the hashish production process, which increases costs and complicates production.

Considering the above, it would be highly desirable to be provided with a hashish product having desired properties while being provided with methods of making same that would at least partially alleviate the disadvantages of the pressing methods discussed above.

### SUMMARY

This Summary is provided to introduce a selection of concepts in a simplified form that are further described below in the Detailed Description. This Summary is not intended to identify key aspects or essential aspects of the claimed subject matter.

Broadly stated, in some embodiments, the present disclosure relates to a process of making a hashish product comprising:
a) providing isolated cannabis trichomes pre-treated to comprise a cannabis oil layer on at least a portion of a surface thereof;
b) mixing the isolated cannabis trichomes under conditions sufficient to obtain a resinous mixture; and
c) retrieving at least a portion of the resinous mixture through an extrusion die to obtain the hashish product comprising a cohesive mass of the isolated cannabis trichomes.

In some embodiments, the process includes one or more of the following features:
the step a) comprises pre-heating the isolated cannabis trichomes under conditions to obtain at least partial decarboxylation of one or more cannabinoid(s) of the isolated cannabis trichomes.
the step a) comprises (i) pre-heating cannabis material under conditions to obtain at least partial decarboxylation of one or more cannabinoid(s) of the cannabis material and (ii) isolating cannabis trichomes therefrom to obtain the isolated cannabis trichomes.
the pre-heating is performed at a temperature of from about 70° C. to about 130° C., preferably from about 80° C. to about 120° C., more preferably about 120° C.
the pre-heating is performed for a duration of from about 10 minutes to about 80 minutes, preferably from about 40 minutes to about 60 minutes, even more preferably from about 50 minutes to about 60 minutes.
the pre-heating is performed to obtain a decarboxylation level of the one or more cannabinoid(s) of from about 30% to about 100%, preferably from about 40% to about 80%, more preferably from about 50% to about 70%, and even more preferably about 60%.
the pre-heating is performed to obtain a ratio of a decarboxylated to acid form content of the one or more cannabinoid(s), by weight, of from about 100:1 to about 1:100, preferably from about 1:4 to about 50:1, more preferably from about 1:3 to about 5:1, even more preferably from about 2:1 to about 4:1, and yet even more preferably about 3:1.
the hashish product comprises a not negligible content in acid form of one or more cannabinoid(s).
the not negligible content in the acid form of the one or more cannabinoid(s) is of no less than 1 wt. %, preferably of no less than 3 wt. %, even more preferably of no less than 5 wt. %.
the pre-heating step is performed on a trichome-containing layer of at least 5 mm.
wherein the trichome-containing layer is no more than 10 mm.
the hashish product comprises a not negligible content in acid form of one or more cannabinoid(s).
the not negligible content in the acid form of the one or more cannabinoid(s) of the hashish product is of no less than 1 wt. %, preferably of no less than 3 wt. %, even more preferably of no less than 5 wt. %.
the not negligible content in the acid form of the one or more cannabinoid(s) of the hashish product is of up to about 30 wt. %, preferably up to about 25 wt. %, more preferably up to about 20 wt. %.
the acid form of the one or more cannabinoid(s) of the hashish product comprises tetrahydrocannabinolic acid (THC-A), cannabidiolic acid (CBD-A), or THC-A and CBD-A.
the pre-heating is performed to obtain a decarboxylation level of the one or more cannabinoid(s) of from about 50% to about 100%, preferably from about 70% to about 100%, more preferably from about 90% to about 100%, and even more preferably about 100%.
further comprising incorporating water to the pre-treated isolated cannabis trichomes prior to step b) to have a water content of about 20 wt. % or less, preferably between about 5 wt. % and about 15 wt. %, more preferably from about 10 wt. % to about 15 wt. %.
the extrusion die is a first extrusion die; and the process comprises passing the resinous mixture through a second extrusion die smaller than the first extrusion die to obtain the hashish product comprising the cohesive mass of the isolated cannabis trichomes.
further comprising cutting the hashish product according to a pre-established cutting operational parameter.
the cutting pattern includes cutting the hashish product along a transverse axis to obtain pieces thereof of substantially identical length and/or weight.
further comprising incorporating one or more additional component(s) prior to, simultaneously with, or following the mixing step.
the one or more additional component(s) includes one or more cannabinoid(s), one or more terpene (s), one or more flavonoid(s), water, one or more flavoring agent(s), one or more non-toxic coloring agent(s), or a mixture thereof.
the one or more cannabinoid(s) is in the form of a crude cannabis extract, a cannabis distillate, a cannabis isolate, a winterized cannabis plant extract, cannabis rosin, cannabis resin, cannabis wax, cannabis shatter, or any combination thereof.
the one or more cannabinoid(s) includes a plurality of cannabinoids.
the one or more cannabinoid(s) includes tetrahydrocannabinol (THC), cannabidiol (CBD), cannabinol (CBN), or any combinations thereof.
further comprising incorporating a crude cannabis extract with the isolated cannabis trichomes.
the crude cannabis extract is incorporated by adding the crude cannabis extract to the isolated cannabis trichomes after the isolated cannabis trichomes are pre-treated to comprise the cannabis oil layer.
the crude cannabis extract is incorporated by adding the crude cannabis extract to the isolated cannabis trichomes before the isolated cannabis trichomes are pre-treated to comprise the cannabis oil layer.
the crude cannabis extract is incorporated prior to the mixing step.
a content of the crude cannabis extract in a combination of the crude cannabis extract and the isolated cannabis trichomes is at least 2 wt. %, preferably at least 5 wt. %, optionally at least 10 wt. %, optionally at least 15%, preferably no more than 20 wt. %, and more preferably between 5 wt. % and 15 wt. %.
said conditions include a selected shear.
said conditions further include a pressure.
comprising pressing the resinous mixture in a press after retrieval through the extrusion die.
the press is capable of applying a pressure of no more than 6000 psi, preferably no more than 5000 psi, more preferably no more than 4000 psi, and even more preferably no more than 3000 psi.
said conditions further include mixing at a selected temperature.
the selected temperature is of about 140° C. or less, preferably between about 20° C. and about 80° C., more preferably about 60° C.
the mixing at the selected temperature is performed for a period of about 5 minutes or more.
the hashish product comprises a cannabinoid content of from about 5 wt. % to about 90 wt. %, preferably from about 10 wt. % to about 60 wt. %, more preferably from about 20 wt. % to about 50 wt. %, even more preferably from about 30 wt. % to about 45 wt. %, and yet even more preferably of about 40 wt. %.
the isolated cannabis trichomes are from a single cannabis strain.
the isolated cannabis trichomes are from a plurality of cannabis strains.
the isolated cannabis trichomes are kief.
the mixing includes applying compression and shear forces to the isolated trichomes via a plurality of interpenetrate helicoidal surfaces within an elongated enclosure.
the elongated enclosure is an extruder device.
the interpenetrate helicoidal surfaces are on at least one screw, the method further comprising adjusting a rotational speed of the at least one screw within the elongated enclosure to obtain the cohesive mass.
the rotational speed of the at least one screw is between about 5 rpm and about 1000 rpm.
the resinous mixture is extruded plural times.

Broadly stated, in some embodiments, the present disclosure relates to a process of making a hashish product, comprising:
a) providing isolated cannabis trichomes pre-heated to comprise a cannabis oil layer on at least a portion of a surface thereof;
b) incorporating a crude cannabis extract with the isolated cannabis trichomes; c) mixing the isolated cannabis trichomes under conditions sufficient to obtain a resinous mixture; and
d) retrieving at least a portion of the resinous mixture through an extrusion die to obtain the hashish product comprising a cohesive mass of the isolated cannabis trichomes.

In some embodiments, the process includes one or more of the following features:
the crude cannabis extract is incorporated by adding the crude cannabis extract to the isolated cannabis trichomes after the isolated cannabis trichomes are pre-heated to comprise the cannabis oil layer.

Broadly stated, in some embodiments, the present disclosure relates to a process of making a hashish product, comprising:
a) providing isolated cannabis trichomes;
b) incorporating a crude cannabis extract with the isolated cannabis trichomes;
c) mixing the isolated cannabis trichomes under conditions sufficient to obtain a resinous mixture; and
d) retrieving at least a portion of the resinous mixture through an extrusion die to obtain the hashish product comprising a cohesive mass of the isolated cannabis trichomes.

In some embodiments, the process includes one or more of the following features:
a content of the crude cannabis extract in a combination of the crude cannabis extract and the isolated cannabis trichomes is at least 15 wt. %.

Broadly stated, in some embodiments, the present disclosure relates to a hashish product made by any process as described herein.

Broadly stated, in some embodiments, the present disclosure relates to a hashish product comprising a cohesive mass of isolated trichomes and having one or more of the following properties as determined in a three-point bending test:
a) higher limit of stiffness of about 3500 g/mm,
b) higher limit of hardness of about 7500 g, and
c) higher limit of toughness of about 25000 g*mm or a lower limit of toughness of about 12000 g*mm.

In some embodiments, the hashish product includes one or more of the following features:
the hashish product includes a not negligible content in acid form of one or more cannabinoid(s).
the hashish product has a lightness value L*≤50 based on a CIELAB scale.
the lightness value L* of the hashish product is no more than about 40, preferably no more than about 30, and more preferably no more than about 20.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments.

### BRIEF DESCRIPTION OF THE DRAWINGS

A detailed description of specific exemplary embodiments is provided herein below with reference to the accompanying drawings in which:

FIG. 1A illustrates a non-limiting flowchart example of a process for obtaining a resinous mixture that is used to obtain an individual unit of hashish product in accordance with an embodiment of the present disclosure.

FIGS. 1B-C illustrate non-limiting flowchart examples of steps for obtaining pre-treated isolated trichomes in accordance with embodiments of the present disclosure.

FIGS. 1D-E illustrate non-limiting flowchart examples of optional steps for incorporating a crude cannabis extract in accordance with embodiments of the present disclosure.

FIG. 1F illustrates a non-limiting flowchart example of a process for working the resinous mixture from FIG. 1A to obtain an individual unit of hashish product in accordance with an embodiment of the present disclosure.

FIG. 1G illustrates a non-limiting flowchart example of a variant of the process in accordance with another embodiment of the present disclosure.

FIG. 2 illustrates a non-limiting system implementing the method of FIG. 1A for manufacturing the hashish product unit.

FIG. 3. illustrates a non-limiting schematic of a setting for performing a density measurement.

FIG. 4 illustrates a non-limiting schematic of a setting for performing a three-point bend test.

FIG. 5 illustrates a non-limiting example of a diagram output from the three-point bend test of FIG. 4.

FIG. 6 illustrates a non-limiting schematic of a setting for performing a puncture test.

FIG. 7 illustrates a non-limiting example of a diagram output from the puncture test of FIG. 6.

FIG. 8 illustrates hashish products corresponding to Example 7.

FIG. 9 illustrates hashish products corresponding to Example 8.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments is provided below along with accompanying figures that illustrate principles of the disclosure. The invention is described in connection with such embodiments, but the invention is not limited to any embodiment. The scope of the invention is limited only by the claims. Numerous specific details are set forth in the following description to provide a thorough understanding of the invention. These details are provided for the purpose of non-limiting examples and the invention may be practiced according to the claims without some or all these specific details. Technical material that is known in the technical fields related to the invention has not been described in detail so that the disclosure is not unnecessarily obscured.

The present inventors have developed a hashish product and methods of manufacturing same that addresses at least some of the above-identified problems.

For example, the present inventors have surprisingly and unexpectedly discovered that mixing pre-treated isolated cannabis trichomes under conditions sufficient to obtain a resinous mixture and retrieving at least a portion of the resinous mixture through an extrusion die alleviates the negative impact of manufacturing hashish products in the batch-like approach discussed previously, while achieving the desired hashish physical attributes, e.g., in terms of malleability, pliability, and/or crumbliness. Indeed, as discussed previously, the existing batch-like approach to manufacturing hash currently requires applying heat during the pressing step via the pressing plates or to the cohesive mass after the pressing step. Further, manufacturers often perform a second pressing step on the cohesive mass after such heat/press step to ensure good cohesiveness of the hashish product. This batch-like multiple steps approach to manufacturing hashish can be labor intensive, reduce volume throughput and negatively affect overall efficiency of the hashish production process, which increases costs and complicates production.

The herein described approach provides the technical advantage of avoiding this batch-like multiple steps approach by pretreating the raw materials instead of the finished product.

For example, large amounts of isolated trichomes or cannabis material can be pre-treated in a single step instead of treating the finished hashish product on a per unit basis, thus avoiding the bottleneck of treating the finished product on a per unit basis.

For example, pre-treatment of isolated trichomes or cannabis material avoids the risk of overcooking the more expensive hashish product that may occur in the batch-like heat treatment of the prior art, as overcooked isolated trichomes or cannabis material may still be used in other applications whereas overcooked hashish products may be less desirable for consumers as the overcooking would likely cause loss of volatile terpenes, thus changing the expected user experience, and/or cause changes to the hashish malleability properties.

For example, pre-treatment of isolated trichomes or cannabis material allows better inventory management as the pre-treated isolated trichomes or cannabis material can be stored for later use and may be used in more than one product type.

Further, it has been observed that implementing the methods of manufacture herein described results in the ability for the operator to control the textural consistency, pliability and/or crumbliness of the hashish product to desired values or ranges of values. Such control thus affords more manufacturing flexibility in terms of producing hashish products with customizable properties, facilitating production runs associated with specific consumer demands, for example.

Further, it has been observed that implementing the method of manufacture herein described results in substantially fewer quality failures (e.g., based on textural consistency, pliability and/or crumbliness), which is advantageous in the context of large-scale industrial production. Such advantages may result in an increase in cost efficiency as well as improve inventory management due to reduction in waste.

Further, existing prior art procedures require each unit of hashish to be manually weighed and trimmed, resulting in non-uniform units and inconsistent appearance. The herein described process allows one to obtain substantially uniform and accurately weighted units since most, if not all, the process can be automated and implemented using mechanical means.

Additionally, or alternatively, controlling the content and homogeneity of the herein described hashish products may allow the manufacturing of hashish products that contain substantially consistent isolated cannabis trichomes components content and distribution therein, within single product units and/or over multiple batches of product units. This in turn can be advantageous in view of increasing consumer demands for predictable user experience.

Further, the herein described hashish product may include not negligible amounts of the acid form of one or more cannabinoid(s). This in turn, may allow the hashish product to have an extended shelf life in comparison to hashish products of the prior art that have fully decarboxylated cannabinoids. Indeed, it is known that the acid form of cannabinoids is more stable over time than decarboxylated form thereof and, as such, cannabinoid potency of the hashish product of the present disclosure can be maintained over extended periods of time comparatively.

Further, the herein described hashish product present substantially consistent homogeneity characteristics, which would be difficult to achieve with known techniques of the known prior art. Such homogeneity characteristics may allow, for example, improvement in the textural consistency, pliability and/or crumbliness of the hashish product. This in turn, may reduce/minimize quality control failures during large-scale manufacturing of the hashish product (e.g., quality control based on textural consistency, pliability and/or crumbliness). Advantageously, it has been observed that such hashish product may afford an enhanced and more consistent user experience in that the reduced crumbliness leads to better segmentation during use of the hashish product that results in reduction of waste material production.

These and other advantages may become apparent to the person of skill in view of the present disclosure.

### Hashish Product

The hashish product of the present disclosure comprises a substantially homogeneous cohesive mass of isolated cannabis trichomes.

By “substantially homogeneous”, it is meant that the hashish product has a constant or uniform composition throughout its cohesive mass.

The level of homogeneity can be measured by detecting proportions of a detectable marker throughout any given sample, allowing for slight measured variations throughout the cohesive mass, e.g., <15% variations, or <10% variations; such slight variations within the cohesive mass will be deemed to be “substantially homogeneous” for the purposes of the present disclosure.

In some embodiments, the detectable marker can be one or more detectable molecule. The one or more detectable molecule may be a component of the isolated trichomes that is detectable using any suitable technique, such as for example Gas Chromatography/Mass Spectrometry (GC/MS), High Pressure Liquid Chromatography (HPLC), Gas Chromatography/Flame Ionization Detection (GC/FID), infra-red spectrum (IR) spectroscopy, ultra-violet spectrum (UV) spectroscopy, Raman spectroscopy, and the like. Other techniques may involve measuring water activity, for example using a capacitive hygrometer (e.g., the AqualabTM 4TE (Meter, USA)) using the chilled-mirror dew point technique, or may involve measuring water content, for example using a moisture analyzer (e.g., MA160 Infrared Moisture Analyzer (Sartorius AG, Germany) using the loss on drying technique (e.g., USP NF 731 Loss On Drying method).

For example, the one or more detectable molecule may be one or more of the following: a cannabinoid, a terpene, a flavonoid, chlorophyll, water, or any combination thereof. Preferably, the detectable molecule is a cannabinoid.

For example, the detectable marker can be detected in at least 90 vol. %, or in at least 95 vol. %, or in at least 99 vol. %, or in 100 vol. % of the hashish product depending on specific implementations of the present disclosure.

Alternatively, or additionally, the levels (or contents) of the detectable marker in the hashish product of the present disclosure is substantially homogeneous, such that the hashish product includes a first content level of the detectable marker in a first discreet portion of the cohesive mass that is within 15% of a second content level of the detectable marker. The second level is an average level of the detectable marker in the hashish product or in a batch of hashish products. For example, the first content level of the detectable marker and the second content level of the detectable marker are present in a ratio first/second content levels of from 0.85 to 1.15. For example, the ratio first/second content levels is of about 0.90, or about 0.95, or about 1.00, or about 1.05, or about 1.10, or about 1.15 or any value therebetween, or in a range of values defined by the aforementioned values. For example, the first discreet portion can be a core portion of the hybrid hash product and the second discreet portion can be a peripheral portion of the hybrid hash product, where the content level of the detectable marker and the ratio of first/second content levels can be determined based on the distribution test described later in this text.

As used herein, the term “cannabis trichomes” generally refers to crystal-shaped outgrowths or appendages (also called resin glands) on cannabis plants typically covering the leaves and buds. Trichomes produce hundreds of known cannabinoids, terpenes, and flavonoids that make cannabis strains potent, unique, and effective.

As used herein, the term “isolated cannabis trichomes” refers to trichomes that have been separated from cannabis plant material using any method known in the art. The details of various methods for separating trichomes from the cannabis plant are well-known in the art. For example, and without wishing to be limiting in any manner, the isolated cannabis trichomes may be obtained by a chemical separation method or may be separated by manual processes like dry sifting or by water extraction methods. Solvent-less extraction methods can include mechanical separation of trichomes from the plant, such as sieving through a screen by hand or in motorized tumblers (see for example WO 2019/161509), or by submerging the cannabis plants in icy water (see for example US2020/0261824, which is herein incorporated by reference) and agitating to separate the trichomes from the plant and drying the trichomes. Because of inherent limitations to existing separation methods, some plant matter or other foreign matter can be present in isolated cannabis trichomes.

Isolated cannabis trichomes obtained by mechanical separation of trichomes from the cannabis plant biomass is typically referred to as “kief” (also “keef” or “kif”) and has a powdery appearance. Typically, some residual plant material remains in the finished kief and thus in the resulting hashish product. In preferred embodiments of the present disclosure, the isolated cannabis trichomes is in the form of kief.

The isolated cannabis trichomes forming the hashish product of the present disclosure may originate from one or more than one strain of cannabis plant. It is known amongst consumers of hashish and other cannabis products that using isolated cannabis trichomes produced from more than one strain of cannabis plant allows a user to tune the psychoactive and/or entourage effect obtained by consuming the product. The mixing of cannabis plant strains may also allow to adjust the final concentration of a component of the product, for example but not limited to the cannabinoid content. Additionally, use of more than one strain allows for improved product and waste management—important in commercial production.

As used herein, the term “cannabis” generally refers to a genus of flowering plants that includes several species. The number of species is currently being disputed. There are three different species that have been recognized, namely Cannabis sativa, Cannabis indica and Cannabis ruderalis. Hemp, or industrial hemp, is a strain of the Cannabis sativa plant species that is grown specifically for the industrial uses of its derived products. In terms of cannabinoids content, hemp has lower concentrations of tetrahydrocannabinol (THC) and higher concentrations of cannabidiol (CBD), which decreases or eliminates the THC-associated psychoactive effects.

The hashish product of the present disclosure comprises one or more cannabinoid(s). As used herein, the term “cannabinoid” generally refers to any chemical compound that acts upon a cannabinoid receptor such as CB1 and CB2. Examples of cannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiolic acid (CBD-A), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), A7-cis-iso tetrahydrocannabivarin, tetrahydrocannabinol (THC, defined below), Δ9-tetrahydrocannabinolic acid (THC-A) including either or both isomers 2-COOH-THC (THCA-A) and 4-COOH-THC (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-A6a-tetrahydrocannabinol, 8,9-dihydroxy-A6a-tetrahydrocannabinol, 10-oxo-A6a-tetrahydrocannabionol (OTHC), 3,4,5,6-tetrahydro-7-hydroxy-a-a-2-trimethyl-9-n-propyl-2,6-methano-2H-1-benzoxocin-5-methanol (OH-iso-HHCV), A6a,10a-tetrahydrocannabinol (A6a,10a-THC), A8-tetrahydrocannabivarin (A8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Patent Application Pub. No. US2014/0271940, which are each incorporated by reference herein in their entirety.

Cannabidiol (CBD) means one or more of the following compounds: A2-cannabidiol, A5-cannabidiol (2-(6-isopropenyl-3-methyl-5-cyclohexen-1-yl)-5-pentyl-1,3-benzenediol); A4-cannabidiol (2-(6-isopropenyl-3-methyl-4-cyclohexen-1-yl)-5-pentyl-1,3-benzenediol); A3-cannabidiol (2-(6-isopropenyl-3-methyl-3-cyclohexen-1-yl)-5-pentyl-1,3-benzenediol); A3,7-cannabidiol (2-(6-isopropenyl-3-methylenecyclohex-1-yl)-5-pentyl-1,3-benzenediol); A2-cannabidiol (2-(6-isopropenyl-3-methyl-2-cyclohexen-1-yl)-5-pentyl-1,3-benzenediol); A1-cannabidiol (2-(6-isopropenyl-3-methyl-1-cyclohexen-1-yl)-5-pentyl-1,3-benzenediol); and A6-cannabidiol (2-(6-isopropenyl-3-methyl-6-cyclohexen-1-yl)-5-pentyl-1,3-benzenediol). In a preferred embodiment, and unless otherwise stated, CBD means A2-cannabidiol.

Tetrahydrocannabinol (THC) means one or more of the following compounds: Δ8-tetrahydrocannabinol (Δ8-THC), Δ8-tetrahydrocannabivarin (Δ8-THCV), Δ9-cis-tetrahydrocannabinol (cis-THC), Δ9-tetrahydrocannabinol (Δ9-THC), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

A cannabinoid may be in an acid form or a non-acid form, the latter also being referred to as the decarboxylated form since the non-acid form can be generated by decarboxylating the acid form.

Advantageously, the hashish product of the present disclosure comprises a content of an acid form of one or more cannabinoid(s) which is not negligible.

As used herein, the term “not negligible” in combination with the concept of the acid form content of the one or more cannabinoids refers to a content which is sufficient to provide the herein described desired physical properties of the hashish product. For example, an acid form content of the one or more cannabinoids which is not negligible can be of no less than about 1 wt. %, preferably of no less than about 3 wt. %, more preferably of no less than about 5 wt. %. For example, the acid form content of the one or more cannabinoids which is not negligible can be of up to about 35 wt. %, up to about 30 wt. %, up to about 25 wt. %, or up to about 20 wt. %, including any values therein or in a range of values defined by the aforementioned values.

The endogenous content of one or more cannabinoid(s) in cannabis strains has been reported in the literature. For example, Coogan in Analysis of the cannabinoid content of strains available in the New Jersey Medicinal Marijuana Program. J Cannabis Res 1, 11 (2019) has reported that various strains of cannabis flower from licensed operators in the New Jersey Medicinal Marijuana Program can be combined in three broad groups of strains: those with <1 wt. % CBD-A and with THC-A concentration from 10 to 30 wt. %; those with both THC-A and CBD-A concentrations in the 5-10 wt. % range; and those with <1 wt. % THC-A and with CBD-A concentration >10 wt. %. It is thus within the reach of the person skilled in the art to use the teachings of the present disclosure to assess the level of decarboxylation required for a given cannabis material or sub-part thereof to obtain the herein desired hashish product properties.

In one embodiment, the hashish product of the present disclosure contains the one or more cannabinoid(s) in an amount sufficient for the user to experience a desired effect when consuming the product. For example, the hashish product may comprise from about 5 wt. % to about 90 wt. % cannabinoid or any value therebetween, or in a range of values defined by any values therebetween. For example, from about 10 wt. % to about 60 wt. %, more preferably from about 20 wt. % to about 50 wt. %. For example, the hashish product may comprise up to about 90 wt. %, up to about 80 wt. %, up to about 70 wt. %, up to about 60 wt. %, or up to about 50 wt. %, or up to about 40 wt. %, or up to about 30 wt. % or any value therebetween, or in a range of values defined by the aforementioned values. For example, the hashish product of the present disclosure may include THC, CBD, CBG, CBN, or any combinations thereof, in similar or different amounts.

In one embodiment, the hashish product may include up to 1000 mg THC per hashish product unit, depending on specific implementations of the present disclosure.

The hashish product can be characterized in several ways, such as in terms of cannabinoid content, terpenes content, water content or physical properties. For example, the hashish product can be characterized in terms of stiffness, hardness, toughness, or a combination thereof, which reflect its malleability, pliability, and/or crumbliness.

As used herein the term “stiffness” refers to the amount of resistance with which a hashish sample opposes a change in the shape under application of a force and is therefore representative of the pliability of the hashish product.

As used herein the term “hardness” refers to the maximum force required for a hashish sample to reach the breaking point and is therefore representative of how easily the hashish product may be cut or separated.

As used herein the term “toughness” refers to the ability of a hashish sample to absorb energy and plastically deform without breaking. Toughness is a measure of the likelihood that the hashish product deforms rather than fractures under an applied force.

In some embodiments, the textural consistency, pliability and/or crumbliness of the hashish product can be characterized with material properties of the product, for example as determined with a three-point bending test using a Texture Analyzer. In such embodiments, the hashish product may be characterized as having one or more of the following: a higher limit of stiffness of about 3500 g/mm as measured in the three-point bend test, a higher limit of hardness of about 7500 g as measured in the three-point bend test, and either a higher limit of toughness of about 25000 g*mm or a lower limit of toughness of about 12000 g*mm as measured in the three-point bend test. In other words, the herein described manufacturing procedure allows customizing the textural properties of the hashish product to desired values, thus facilitating responding to market demand.

For example, the stiffness can be of from about 200 g/mm to about 3500 g/mm, including any ranges there in-between or any values therein. For example, the stiffness can be of about 200 g/mm, about 250 g/mm, about 300 g/mm, about 350 g/mm, about 400 g/mm, about 450 g/mm, 500 g/mm, about 550 g/mm, about 600 g/mm, about 650 g/mm, about 700 g/mm, about 750 g/mm, about 800 g/mm, about 850 g/mm, about 900 g/mm, about 950 g/mm, about 1000 g/mm, about 1050 g/mm, about 1100 g/mm, about 1150 g/mm, about 1200 g/mm, about 1250 g/mm, about 1300 g/mm, about 1350 g/mm, about 1400 g/mm, about 1450 g/mm, about 1500 g/mm, about 1550 g/mm, about 1600 g/mm, about 1650 g/mm, about 1700 g/mm, about 1750 g/mm, about 1800 g/mm, about 1850 g/mm, about 1900 g/mm, about 1950 g/mm, about 2000 g/mm, about 2100 g/mm, about 2200 g/mm, about 2300 g/mm, about 2400 g/mm, about 2500 g/mm, about 2600 g/mm, about 2700 g/mm, about 2800 g/mm, about 2900 g/mm, about 3000 g/mm, about 3100 g/mm, about 3200 g/mm, about 3300 g/mm, about 3400 g/mm, or about 3500 g/mm, including any value there in-between or any ranges with any of these values as range limits.

For example, the hardness can be of from about 250 g to about 7500 g, including any ranges there in-between or any values therein. For example, the hardness can be of about 250 g, about 500 g, about 750 g, about 1000 g, about 1150 g, about 1200 g, about 1250 g, about 1300 g, about 1350 g, about 1400 g, about 1450 g, about 1500 g, about 1550 g, about 1600 g, about 1650 g, about 1700 g, about 1750 g, about 1800 g, about 1850 g, about 1900 g, about 1950 g, about 2000 g, about 2100 g, about 2200 g, about 2300 g, about 2400 g, about 2500 g, about 2600 g, about 2700 g, about 2800 g, about 2900 g, about 3000 g, about 3500 g, about 4000 g, about 4500 g, about 5000 g, about 6000 g, about 6500 g, or about 7500 g, including any value there in-between or any ranges with any of these values as range limits.

For example, the toughness can be of from about 1500 g*mm to about 12500 g*mm, including any ranges there in-between or any values therein. For example, the toughness can be of about 1500 g*mm, about 2500 g*mm, about 3500 g*mm, about 4500 g*mm, about 5500 g*mm, about 6500 g*mm, about 7500 g*mm, or about 8500 g*mm, including any value there in-between or any ranges with any of these values as range limits.

For example, the toughness can be of from about 12500 g*mm to about 25000 g*mm including any ranges there in-between or any values therein. For example, the toughness can be of about 12500 g*mm, about 13000 g*mm, about 13500 g*mm, about 14000 g*mm, about 14500 g*mm, about 15000 g*mm, about 15500 g*mm, about 16000 g*mm, about 16500 g*mm, about 17000 g*mm, about 17500 g*mm, about 18000 g*mm, about 18500 g*mm, about 19000 g*mm, about 19500 g*mm, about 20000 g*mm, about 20500 g*mm, about 21000 g*mm, about 21500 g*mm, about 22000 g*mm, about 22500 g*mm, about 23000 g*mm, about 23500 g*mm, about 24000 g*mm, about 24500 g*mm, or about 25000 g*mm, including any value there in-between or any ranges with any of these values as range limits.

In some embodiments, the moisture content in the hashish product of the present disclosure, achieved through addition of water to the isolated trichomes, can be of about 5 wt. % or more. For example, the moisture content can be of from 10 wt. % to about 50 wt. %, or any value therebetween, or in a range of values defined by any values therebetween, as described in PCT Application PCT/CA2020/051733, which is hereby incorporated by reference in its entirety.

### Additional Components

The hashish product according to the present disclosure may also comprise one or more additional components.

In some embodiments, the one or more additional components may be added to alter the characteristics of the hashish product, such as cannabinoid content, potency, entourage effect, odor, color, consistency, texture, pliability, and the like.

In some embodiments, the one or more additional components may be incorporated throughout the hashish product, or the one or more additional components may be distributed on at least a portion of a surface of the hashish product, for example as a coating. In some embodiments, the one or more additional components may be substantially homogeneously distributed on the at least portion of the surface of the hashish product. By “substantially homogeneously distributed”, it is meant that the amount of the one or more additional component is uniform on the at least portion of the surface of the hashish product.

The one or more additional components may be any suitable food grade and/or non-toxic composition or component known in the art. As will be recognized by those of skill in the art, the toxicity of each type of additional component may be dependent on the method of consumption of the hashish product. For example, in applications where smoke/vapor produced by the hashish product is to be inhaled, suitable additional components may include, but are not limited to one or more cannabinoid, one or more terpene (also referred to herein as a “terpene blend”), one or more flavonoid, water, or any combination thereof.

The one or more additional components may be a cannabinoid. The cannabinoid may be extracted from any suitable source material including, but not limited to, cannabis or hemp plant material (e.g., flowers, seeds, and trichomes) or may be manufactured artificially (for example cannabinoids produced in yeast, as described in WO WO2018/148848). Cannabinoids can be extracted from a cannabis or hemp plant material according to any procedure known in the art. For example, and without wishing to be limiting, a “crude extract” containing a cannabinoid may be obtained by extraction from plant materials using for example aliphatic hydrocarbons (such as propane, butane), alcohols (such as ethanol), petroleum ether, naphtha, olive oil, carbon dioxide (including supercritical and subcritical CO₂), chloroform, or any combinations thereof. Optionally, the crude extract may then be “winterized”, that is, extracted with an organic solvent (such as ethanol) to remove lipids and waxes (to produce a “winterized extract”), as described for example in U.S. Pat. No. 7,700,368, US 2004/0049059, and US 2008/0167483, which are each herein incorporated by reference in their entirety. Optionally, the method for obtaining the cannabinoid may further include purification steps such as a distillation step to further purify, isolate or crystallize one or more cannabinoids, which is referred to in the art and herein as a “distillate”; US20160346339, which is incorporated herein by reference, describes a process for extracting cannabinoids from cannabis plant material using solvent extraction followed by filtration, and evaporation of the solvent in a distiller to obtain a distillate. The distillate may be cut with one or more terpenes. The crude extract, the winterized extract or the distillate may be further purified, for example using chromatographic and other separation methods known in the art, to obtain an “isolate”. Cannabinoid extracts may also be obtained using solvent-less extraction methods; for example, cannabis plant material may be subjected to heat and pressure to extract a resinous sap (“rosin”) containing cannabinoids; methods for obtaining rosin are well-known in the art.

The one or more additional components may be a terpene. As used herein, the term “terpene” generally refers to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Any suitable terpene may be used in the hashish product of the present invention. For example, terpenes originating from cannabis plant may be used, including but not limited to aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, hashishene, and derivatives thereof. Further examples of terpenes are discussed in US Patent Application Pub. No. US2016/0250270, which is herein incorporated by reference in its entirety for all purposes. The hashish product of the present disclosure may contain one or more terpene(s). The one or more terpene(s) may originate from the hashish, from an additional component, or both. In some embodiments, the hashish product of the present disclosure may include the one or more terpene(s) in an amount (the “terpene content”) sufficient for the user to experience a desired entourage effect when consuming the product. For example, the hashish product may comprise from about 0.5 wt. % to about 15 wt. % terpene, for example up to about 15 wt. %, or up to about 10 wt. %, or up to about 5 wt. %, or up to about 4 wt. %, or up to about 3 wt. %, or up to about 2 wt. %, or up to about 1 wt. %. For example, the one or more terpene(s) may include hashishene. Without wishing to be bound by theory, hashishene is believed to be a terpene produced by rearrangement of myrcene that may be found in hashish after mechanical processing, and that may be responsible for the typical desirable “hashish flavour”.

The one or more additional components may be a flavonoid. The term “flavonoid” as used herein refers to a group of phytonutrients comprising a polyphenolic structure. Flavonoids are found in diverse types of plants and are responsible for a wide range of functions, including imparting pigment to petals, leaves, and fruit. Any suitable flavonoid may be used in the hashish product of the present invention. For example, flavonoids originating from a cannabis plant may be used, including but not limited to: apigenin, cannflavin A, cannflavin B, cannflavin C, chrysoeril, cosmosiin, flavocannabiside, homoorientin, kaempferol, luteolin, myricetin, orientin, quercetin, vitexin, and isovitexin.

The reader will readily understand that in some embodiments, the one or more components may include any combinations of the herein described one or more component(s).

### Methods of Using Hashish

Hashish products are typically used for recreational or medicinal purposes. For example, hashish can be used to achieve a desired effect in a user, such as a psychoactive effect, a physiological effect, or a treatment of a condition. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of cannabinoid(s) at sufficient amounts to mediate the therapeutic effects.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a mammal, such as a dog, cat or human, preferably a human.

In certain embodiments, the disease or condition is selected from the group consisting of pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction.

In the context of recreational use, the “effective amount” administered and rate and time-course of administration, will depend on the desired effect associated with a feeling of physical and/or emotional satisfaction in the subject.

In the context of health and wellness use, the “effective amount” administered and rate and time-course of administration will depend on the nature and severity of the disease or condition being treated and typically also takes into consideration the condition of the individual subject, the method of administration and the like.

### Manufacturing Process

The hashish product may be produced by mechanically mixing the components thoroughly to provide a substantially homogeneous resinous mixture. By the term “mechanically mixing” or “mechanical mixing”, it is meant mixing using any suitable mechanical means. The mechanical means may be a plurality of interpenetrate helicoidal surfaces within an elongated enclosure or barrel, a non-limiting example of which is an extruder apparatus.

An extruder is a machine used to perform the extrusion process. Manufacturing by extrusion occurs when a material (usually pellets, dry powder, rubber, plastic, metal bar stock or food) is heated and pushed through a die assembly. A die is a mold that shapes the heated material as it is forced through a small opening from the inside of the extruder to the outside. Using a system of barrels or cylinders containing interpenetrate helicoidal surfaces, e.g., screw pumps or extruder screws, the extruder can mix the ingredients while heating and propelling the extrudate through the die to create the desired shape.

An extruder can have a single extruder screw or twin extruder screws, and can be configured to have one or more mixing zones, one or more temperature zones, and one or more input zones. The input zones are used for introduction of material. The mixing zones apply compression and shear forces to the input materials, blending until they are homogenized. The extruder die assembly may perform a variety of functions: it may form or shape the extrudate, it may divide the extrudate into multiple extrudates, it may inject one or more component into the extrudate, and it may compress and reduce the cross-sectional area of the extrudate.

Single screw extruders are known in the art—the screws of such extruders comprise grooves and may be cylindrical, conical, tapered and the likes as described for example in CA 2,731,515, U.S. Pat. No. 6,705,752, CN101954732 and CN201792480, where each of which is herein incorporated by reference in its entirety. Twin screw extruders are also know in the art—screws of such extruders may be parallel or non-parallel, converging or non-converging, with or without differential speed, counter or non-counter rotating as described for example in U.S. Pat. No. 6,609,819, WO 2020/220390, WO 2020/220495 and US 2010/0143523, where each of which is herein incorporated by reference in its entirety. Single screw and twin screw arrangements may also be integrated within a single extruder device, as described for example in U.S. Pat. No. 10,124,526, which is herein incorporated by reference in its entirety. It will be readily appreciated that extruders have flexible configuration (in terms of mixing zones, temperature zones, input zones, etc.) and that any suitable configuration of the extruder apparatus that produces the hash product may be used within the context of the present disclosure.

The mechanical mixing can be applied to the isolated cannabis trichomes within the extruder under conditions sufficient to obtain a cohesive, continuous, and substantially homogenous resinous mixture. The conditions or variables that can be modified during production are discussed later in this text.

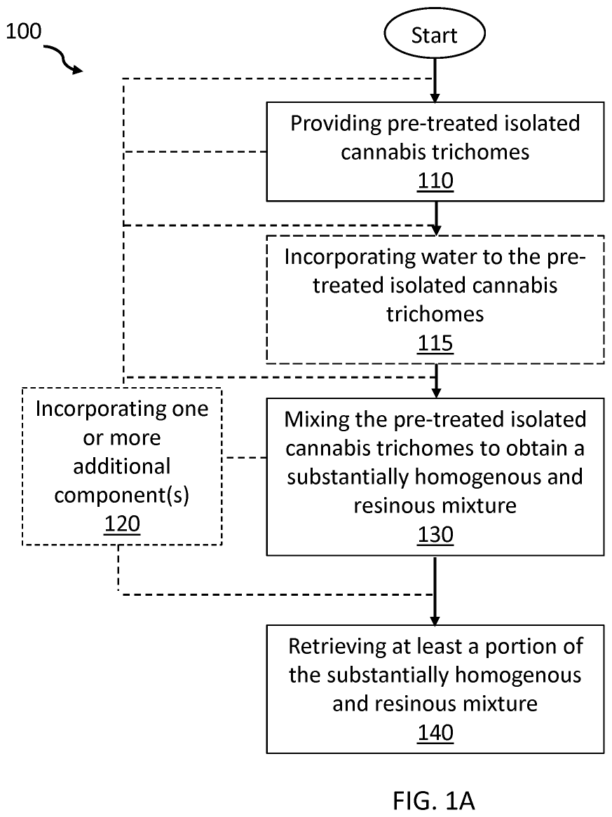

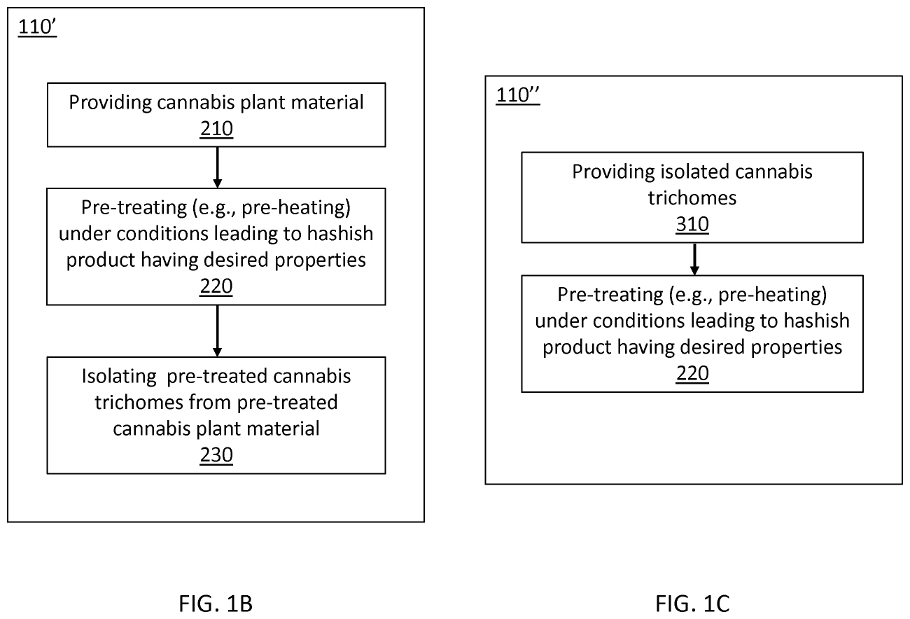

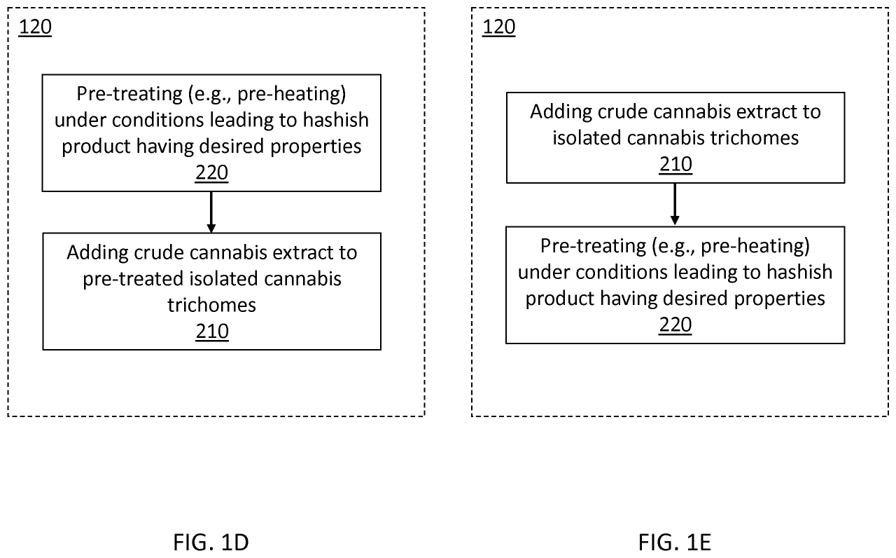

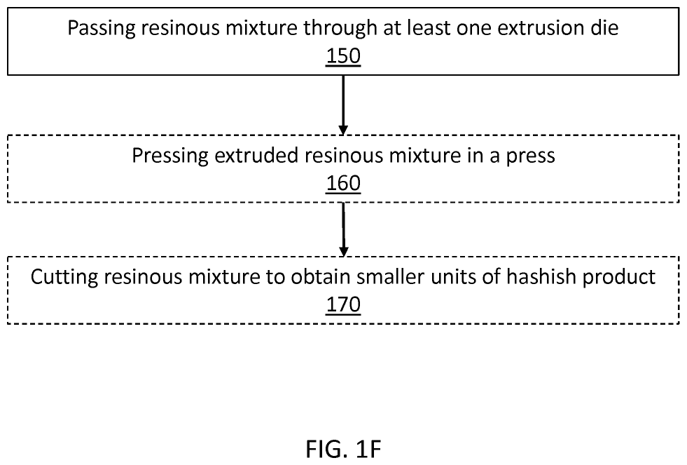

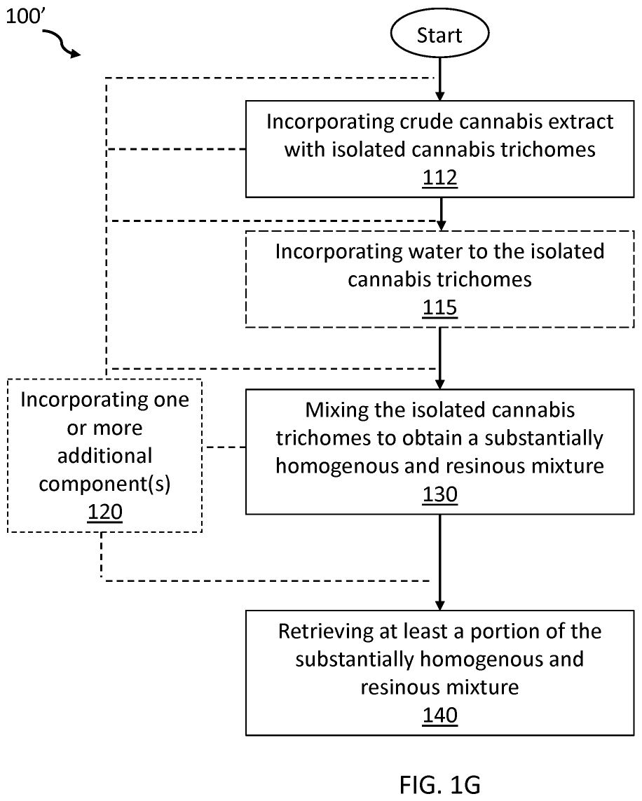

FIG. 1A is a flowchart of a general process 100 for making a hashish product in accordance with an embodiment of the present disclosure. The process 100 comprises a first step 110 of providing pre-treated isolated cannabis trichomes (alone or together with one or more additional components as will be described later in this text).

In one non-limiting example, the pre-treated isolated cannabis trichomes may include trichomes isolated from a single cannabis strain. In another non-limiting example, the pre-treated isolated cannabis trichomes may include trichomes isolated from a plurality of distinct cannabis strains, which may have different respective cannabinoid(s) and/or terpene(s) content. The choice of one over the other may be driven by practical considerations, such as but not limited to inventory management considerations, the desired cannabinoid content of the hashish product, the desired user experience, and the like. It is known amongst consumers of hashish and other cannabis products that using isolated cannabis trichomes produced from more than one strain of cannabis plant may allow a user to tune the psychoactive and/or entourage effect obtained by consuming the product. The mixing of cannabis plant strains may also allow adjustments to the final concentration of a component of the product, for example but not limited to the cannabinoid content. Additionally, use of more than one strain allows for improved product and waste management—important in commercial production. The isolated cannabis trichomes can be kief.

The pre-treated isolated cannabis trichomes may be obtained in several ways.

The producer implementing the process 100 may obtain the pre-treated isolated cannabis trichomes from another producer. The step 110 may thus include a sub-step of obtaining the pre-treated isolated cannabis trichomes from another producer (not shown in figures).

Alternatively, the producer implementing the process 100 may obtain the pre-treated isolated cannabis trichomes via at least one of the following variants of step 110.

FIG. 1B is a first variant step 110′ which includes starting from pre-treated cannabis plant material to isolate the pre-treated cannabis trichomes therefrom. In this variant, a first step 210 includes providing cannabis plant material comprising cannabis trichomes. The cannabis plant material may comprise cannabis flowers/buds, cannabis trim, cannabis leaves, or any combination thereof. The producer implementing the first variant step 110′ may also produce the cannabis plant material or may obtain the cannabis plant material from another producer. In a second step, the cannabis plant material is pre-treated with a pre-heating step 220 under conditions leading to ultimately obtaining a hashish product with the desired properties, such as in terms of malleability, crumbliness, and/or pliability. In a subsequent step 230, cannabis trichomes are isolated from the pre-treated cannabis plant material thus resulting in the pre-treated isolated cannabis trichomes. As discussed previously, various processes for isolating cannabis trichomes from cannabis plant material are known and as such, will not be further described here.

FIG. 1C is a second variant step 110″ which includes starting from isolated cannabis trichomes to obtain the pre-treated isolated cannabis trichomes. In this variant, a first step 310 includes providing isolated cannabis trichomes. The producer implementing the second variant step 110″ may also produce the isolated cannabis trichomes or may obtain the isolated cannabis trichomes from another producer. In a second step, the isolated cannabis trichomes are pre-treated with the pre-heating step 220 described above.

The present inventors have surprisingly discovered that the conditions for performing the pre-heating step 220 can be optimized using the decarboxylation level as a biomarker to monitor the extent of pre-heating that is suitable for a given situation. Indeed, the present inventors have discovered that when the pre-heating step 220 is performed under conditions leading to at least partial decarboxylation (i.e., partial, near complete, or complete decarboxylation), one can ultimately obtain a hashish product having the desired properties, such as in terms of malleability, crumbliness, and/or pliability. For example, in some embodiments, the pre-heating step 220 may be performed under conditions such that the content in acid form of one or more cannabinoid(s) of the pretreated material (i.e., cannabis plant material or isolated cannabis trichomes) is not negligeable.

In some embodiments, the variant steps 110′ and/or 110″ may be performed at a first location while the remaining steps of process 100 may be performed at a second location, where the first and second locations may be within the same licensed producer site or within different licensed producer sites. In some embodiments, all steps of process 100 may be performed at the same location.

The pre-treatment of the isolated cannabis trichomes can be monitored in several ways.

In some embodiments, the pre-treatment of the isolated cannabis trichomes may be performed to obtain a desired decarboxylation level. For example, the decarboxylation level can be from about 30% to about 100%, or any value therebetween, or in a range of values defined by any values therebetween. For example, in some embodiments, the level of decarboxylation may be from about 40% to about 80%, from about 50% to about 70%, or about 60%. As another example, in some embodiments, the level of decarboxylation may be from about 50% to about 100%, from about 70% to about 100%, from about 90% to about 100%, or about 100%.

The decarboxylation level can be determined, for example, by comparing the initial content in the acid form of a specific cannabinoid to the remaining content in the acid form of the specific cannabinoid after the pre-heating step 220. For example, if the initial content in the acid form of a specific cannabinoid is 30 wt. % and the remaining content in the acid form of that specific cannabinoid obtained after the pre-heating step 220 is 15 wt. %, it means that the decarboxylation level is 50% (i.e., half of the initial content in the acid form of a specific cannabinoid was decarboxylated).

In some embodiments, the pre-treatment of the isolated cannabis trichomes may be performed to obtain a desired ratio of decarboxylated vs. acid form (wt. %:wt. %) of a specific cannabinoid. For example, the ratio may be of from about 100:1 to about 1:100, or any value therebetween, or in a range of values defined by any values therebetween. For example, in some embodiments, from about 1:4 to about 50:1, or from about 1:3 to about 5:1, even more preferably from about 2:1 to about 4:1, and yet even more preferably about 3:1. As another example, in some embodiments, from about 80:1 to about 100:1, from about 90:1 to about 100:1, or from about 95:1 to about 100:1.

The content in the acid form and the decarboxylated form of a specific cannabinoid can be determined using suitable methods known to the person skilled in the art, such as but not limited to Gas Chromatography/Mass Spectrometry (GC/MS), High Performance Liquid Chromatography (HPLC), Gas Chromatography/Flame Ionization Detection (GC/FID), Fourier transform infrared (FT-IR) spectroscopy, and the like. Various suitable methods are described, for example, in Formato et al., “(−)-Cannabidiolic Acid, a Still Overlooked Bioactive Compound: An Introductory Review and Preliminary Research.” Molecules. 2020 Jun. 5; 25(11):2638.

The process 100 may further comprises an optional step 115 of incorporating water to the pre-treated isolated cannabis trichomes prior to the mixing step, as further described below. Water may be incorporated in the form of steam, liquid, ice, or a combination. The water incorporated may be distilled, reverse osmosis and/or microfiltered water. In some embodiments, water may be incorporated to have a total water content of about 20 wt. % or less. For example, a total water content of from about 5 wt. % to about 15 wt. % or any value therebetween, or in a range of values defined by any values therebetween. For example, a total water content of about 15 wt. % or less, about 14 wt. % or less, about 13 wt. % or less, about 12 wt. % or less, about 11 wt. % or less, about 10 wt. % or less. For example, a total water content of from about 10 wt. % to about 15 wt. %, from about 10 wt. % to about 12 wt. %.

It will be readily appreciated that the total water content of the isolated cannabis trichomes may be adjusted to any desired/target value. The relative amount of water being incorporated into the pre-treated isolated cannabis trichomes at optional step 115 may be dependent upon several factors, as further described below, such as the extrusion conditions, the conditions for performing the pre-heating step 220 and/or the desired physical properties of the hashish product.

It is further noted that, in some embodiments, the optional step 115 may only be performed when the step of pre-heating the isolated cannabis trichomes is done for a shorter period of time (e.g., 25-30 minutes) at a temperature of about 120° C.

It is to be understood that, in some embodiments, the optional step 115 is not performed, i.e., no water is added to the pre-treated isolated cannabis trichomes.

In some embodiments, the conditions for performing the pre-heating step 220 may include time duration, temperature, or a combination thereof.

For example, the pre-heating temperature may be from about 70° C. to about 130° C., or any value therebetween, or in a range of values defined by any values therebetween. For example, from about 80° C. to about 120° C., or about 120° C.

For example, the pre-heating time may be from about 10 minutes to about 80 minutes, or any value therebetween, or in a range of values defined by any values therebetween. For example, from about 40 minutes to about 60 minutes, or about 45 minutes, or about 50 minutes, or about 55 minutes. For example, from about 50 minutes to about 60 minutes.

It will be readily apparent to the person skilled in the art that various combinations and permutations of pre-heating temperature and pre-heating time may be used to achieve identical, similar or substantially similar decarboxylation content of a specific cannabinoid (e.g., a longer pre-heating time with a lower pre-heating temperature, a higher pre-heating temperature with a shorter pre-heating time, etc.) and it is well within the skills of that person to select and implement such combinations and permutations that will achieve the desired result, in view of the herein described teachings. For example, it will be also readily apparent to the person skilled in the art that the pre-heating time and the pre-heating temperature may be selected based on the strain of cannabis plant, the type of cannabis plant material (where applicable), the method of isolation of the cannabis trichomes (where applicable), and the like.

In some non-limiting examples, the pre-heating temperature can be of about 120° C. and the pre-heating time may be of from about 10 to about 80 minutes; in some cases, the pre-heating temperature can be of about 120° C. and the pre-heating time may be of from about 40 to about 60 minutes, or from about 50 to about 60 minutes. In other non-limiting examples, the pre-heating temperature and/or the pre-heating time may have other suitable values. For instance, in some cases, the pre-heating temperature can be of about 100° C. and the pre-heating time may be of from about 60 to about 80 minutes; in some cases, the pre-heating temperature can be of about 80° C. and the pre-heating time may be of from about 120 minutes or more; or any other range or value within such ranges.

In some embodiments, the pre-heating step 220 may be performed in any suitable heating apparatus (e.g., an oven), which may be equipped with proper ventilation if desired.

In some embodiments, the pre-heating step 220 may be performed on a trichome-containing layer, which is a layer of the isolated cannabis trichomes or of the cannabis plant material from which the cannabis trichomes are isolated, and which has been layered onto a plaque or other support for heating by the heating apparatus. For example, in some embodiments, a thickness of the trichome-containing layer may be at least 5 mm and/or no more than 10 mm (e.g., as this may assist in proper heat penetration throughout). The thickness of the trichome-containing layer may have any other suitable value in other embodiments. For example, in some embodiments, the thickness of the trichome-containing layer may be greater (e.g., more than 10 mm) by modulating timing and duration of heating.

In some embodiments, the herein described acid form of the one or more cannabinoid(s) comprises THC-A, CBD-A, or a combination. Preferably, the specific cannabinoid assessed to monitor the level of decarboxylation described herein is THC-A.

### Mixing Pre-Treated Trichomes and Retrieving Through a Die

Returning to FIG. 1A, the process 100 further comprises a step 130 of mixing the pre-treated isolated cannabis trichomes. Such mixing may be performed mechanically with an extruder, for example. The pre-treated isolated cannabis trichomes are mixed under conditions sufficient to obtain a substantially homogenous and resinous mixture.

The conditions to form the cohesive mass of the pre-treated isolated cannabis trichomes at the mixing step 130 comprise shear and/or pressure, and optionally temperature, which may be varied to alter the characteristics of the hashish product. Such characteristics may include, but without being limited to, stiffness (i.e., characteristic that defines the level of malleability of the hashish product), hardness or resistance to localized deformation (i.e., characteristic that determines how easy it is to cut or separate the hashish product), toughness (i.e., characteristic that determines the likelihood that the hashish product deforms rather than fractures under an applied force), color, tactual characteristics, and the like.

For example, the pressure being applied at the mixing step 130 may be at a value of about 5 psi or more. For example, a pressure of from about 5 psi to about 500 psi, including any ranges therein or any value therein. For example, a pressure of from about 5 psi to about 300 psi, from about 20 psi to about 300 psi, or from about 20 psi to about 250 psi, including any ranges therein or any value therein. For example, a pressure of about 20 psi, about 30 psi, about 40 psi, about 50 psi, about 100 psi, about 150 psi, about 200 psi, about 250 psi, about 300 psi. The person of skill will readily understand that a given pressure value may be obtained depending on the die and/or the mixing rotor speed that is used to form the hashish product, as described elsewhere in this text.

For example, the pressure being applied at the mixing step 130 may be performed for a time of about 0.5 minutes (30 seconds) or more. When implementing the herein described process in an elongated enclosure, such as an extruder, the pressure being applied at the mixing step 130 will be performed for a time that will vary at least based on the length of the enclosure and processing speed through the length of the enclosure. For example, the pressure being applied at the mixing step 130 may be performed for a time of from about 0.5 (30 seconds) to about 60 minutes, including any ranges therein or any value therein. For example, a time of about 5 minutes, about 10 minutes, about 15 minutes, about 20 minutes, about 30 minutes, about 40 minutes, about 50 minutes, or about 60 minutes.

For example, the temperature being applied at the mixing step 130 may be at a value of about 140° C. or less. For example, a temperature of from about 20° C. to about 120° C., including any ranges therein or any value therein. For example, a temperature of about 20° C., about 30° C., about 40° C., about 50° C., about 60° C., about 70° C., about 80° C., about 90° C., about 100° C., about 110° C., about 120° C., about 130° C., or about 140° C. In some practical implementations, the temperature at the mixing step 130 may be monitored in-process using a live temperature probe, for example.

For example, the temperature being applied at the mixing step 130 may be performed for a period of about 0.5 minutes (30 seconds) or more. When implementing the herein described process in an elongated enclosure, such as an extruder, the temperature being applied at the mixing step 130 will be performed for a time that will vary at least based on the length of the enclosure and processing speed through the length of the enclosure. For example, the temperature being applied at the mixing step 130 may be performed for a time of from about 0.5 (30 seconds) to about 60 minutes, including any ranges therein or any value therein. For example, a time of about 5 minutes, about 10 minutes, about 15 minutes, about 20 minutes, about 30 minutes, about 40 minutes, about 50 minutes, or about 60 minutes.

In one practical implementation, the mixing includes applying compression and shear forces to the isolated cannabis trichomes via a plurality of interpenetrate helicoidal surfaces within an elongated enclosure. Preferably, the elongated enclosure is an extruder device having at least one screw. The mixing shear and compressive forces can be controlled by modulating the rotational speed of at least one of the screws within the extruder. In such embodiments, the extruder screw rotation per minute (rpm) can be selected to perform the mixing step 130 at a value of for example about 5 rpm or more. For example, the extruder screw rpm can be selected in a range of from about 5 rpm to about 1000 rpm, including any ranges therein or any value therein. For example, from about 15 to about 500 rpm, or from about 25 to about 450 rpm, or from about 30 to about 400 rpm, or from about 45 to about 450 rpm including any value within any of these ranges. In such embodiment, the pressure applied by the extruder screw can be accompanied by heat to enhance mixing of the isolated cannabis trichomes, extract the resinous content of the trichomes and obtain a heated, cohesive, continuous, and substantially homogenous resinous mixture. In such embodiment, the heating and mixing can continue until a desired level of homogeneity is obtained. For example, a time of about 5 minutes, about 10 minutes, about 15 minutes, about 20 minutes, about 30 minutes, about 40 minutes, about 50 minutes, or about 60 minutes. In some embodiments, the heating and mixing continues until the desired level of homogeneity is determined by testing samples of mass retrieved from the process.

In embodiments where the heating and mixing are performed in a single screw extruder, the residence time within the extruder barrel can be directly related to the length of the barrel and the rotational speed of the single screw. To increase mixing time of the components within the barrel, the components can travel through the length of the barrel, and then be redirected to the inlet (rather than proceed through the die).

Optional step 120 includes incorporating one or more additional component(s) at one or more step(s) during the process 100. For example, one or more additional component(s) can be added to the isolated trichomes prior to, simultaneously with, or following step 110, or prior to, simultaneously with, or following the mixing step 130. Multiple additional components may be added in a single step or may be added separately in one or more consecutive steps or at different times or points along the process 100. The one or more additional components can be one or more cannabinoids, one or more terpenes, one or more flavonoids, water, one or more flavoring agents, one or more non-toxic coloring agents, or any combination thereof. The person of skill will readily appreciate that water could be added in the form of steam, liquid, ice, or in any combination thereof. When the one or more component comprises a cannabinoid, the cannabinoid may be provided in the form of a cannabis extract (including a crude extract, or a winterized extract), a distillate, an isolate, cannabis rosin, cannabis resin, cannabis wax, or cannabis shatter.

In some embodiments, the one or more additional component may be incorporated during the process to produce the hashish product and thus may be substantially homogeneously distributed throughout the hashish product. Alternatively, or additionally, the one or more additional component may be substantially homogenously distributed on at least a portion of a surface of the hashish product, for example as a coating. For example, the portion of the surface of the hashish product may include at least 20%, at least 30%, at least 40%, at least 50%, at least 60%, at least 70%, at least 80%, at least 90%, or 100% of the surface of the hashish product. By “substantially homogeneously distributed”, it is meant that the amount of the one or more additional component is uniform on the at least portion of the surface of the hashish product.

In some embodiments, the one or more cannabinoids can be in extracted and purified form and may include a crude cannabis extract, a cannabis distillate, a cannabis isolate, a winterized cannabis extract, cannabis rosin, cannabis resin, cannabis wax, or cannabis shatter, or any possible combination thereof.

For example, in some embodiments, as part of the optional step 120, a crude cannabis extract (e.g., a crude cannabis oil having a cannabinoid content of no more than 80 wt. %, such as between 50 wt. % and 80 wt. %) may be incorporated with the isolated cannabis trichomes to be part of the hashish product. This may facilitate mechanical processing by a mixing apparatus (e.g., the extruder) in the mixing step 130 (e.g., which may be particularly useful where the mixing apparatus is a low-shear one, such as by reducing passes) and/or enhance organoleptic and/or other physical properties of the hashish product (e.g., with higher terpene content). With infusion of the crude cannabis extract, the hashish product may be referred to as a “crude-infused” hashish product.

In some embodiments, as shown in FIG. 1D, the crude cannabis extract may be incorporated by adding it to the pre-treated isolated cannabis trichomes, i.e., by adding the crude cannabis extract to the isolated cannabis trichomes after they have been pre-heated. In such cases, this may have a synergistic effect with pre-heating of the isolated cannabis trichomes that can be beneficial for mechanical processing.

In other embodiments, as shown in FIG. 1E, the crude cannabis extract may be incorporated by adding it to the isolated cannabis trichomes before their pre-heating. Thus, in such embodiments, the crude cannabis extract may be added to the isolated cannabis trichomes, a resulting mix of the crude cannabis extract and the isolated cannabis trichomes can be pre-heated, and then that pre-heated mix is processed and extruded in subsequent steps as discussed above. In some examples, the crude cannabis extract may itself be pre-heated and then added to the isolated cannabis trichomes, which in some cases may contribute to heating the isolated cannabis trichomes.

For example, in some embodiments, a content of the crude cannabis extract in a combination of the crude cannabis extract and the isolated cannabis trichomes may be at least 2 wt. %, in some cases at least 5 wt. %, in some cases at least 10 wt. %, and in some cases at least 15%. In some examples, the content of the crude cannabis extract in the combination of the crude cannabis extract and the isolated cannabis trichomes may be no more than 20 wt. %. For instance, in some cases, the content of the crude cannabis extract in the combination of the crude cannabis extract and the isolated cannabis trichomes may be between 5 wt. % and 15 wt. %.

In some embodiments, the one or more terpenes may include one or more terpenes which are endogenous to the cannabis strain or plurality of cannabis strains from which stem the isolated cannabis trichomes. The one or more terpenes may include one or more terpenes that are not naturally found in the one or more cannabis strain(s) from which stem the isolated cannabis trichomes.

Once the substantially homogenous and resinous mixture is obtained at step 130, at least a portion of the substantially homogenous and resinous mixture is retrieved at step 140 to obtain an individual unit of hashish product having a cohesive mass of the isolated trichomes.

FIG. 1F includes one or more additional steps that can be part of and/or follow step 140 of the process 100 in FIG. 1A.

For example, the portion of the substantially homogenous and resinous mixture can be passed through one or more dies (e.g., extrusion dies) at step 150, which may be configured to impart one or more pre-determined shapes to the resinous mixture. Another effect of passing through one or more dies is that the die(s) additionally impart(s) shear/pressure to the substantially homogenous and resinous mixture that results in releasing more resin from the isolated cannabis trichomes which eventually improves binding the entire body of hashish product. In some embodiments, the resinous mixture may be extruded plural times (i.e., undergo two or more extrusion passes) and/or differently-sized extrusion dies (i.e., having die openings of different sizes) may be used one after another to modulate shearing actions and product characteristics (e.g., color, malleability, etc.). For example, in some cases, a larger extrusion die (e.g., with an opening of a ¼ in.) may be used for one pass followed by a smaller extrusion die (e.g., with an opening of 5/32 in.) for another pass.

In some embodiments, at optional step 160, the resinous mixture that was extruded may be pressed in a press. This may allow the extruded resinous mixture to be shaped into a block of another pre-determined shape that is different from the pre-determined shape imparted during extrusion of the resinous mixture. In turn, this may facilitate forming the hashish products into desired final shapes (e.g., rectangular strips, bricks or other square-edged products, etc.). Because the extruded resinous mixture is more malleable, less pressing may be involved. For example, in some embodiments, the press may be a low-pressure press and/or fewer pressing steps (e.g., a single pressing step) may be used, which may be more cost-effective and/or efficient by using pressing equipment that is less expensive and/or less time. The low-pressure press may be capable of applying a pressure of no more than 6000 psi, in some cases no more than 5000 psi, in some cases no more than 4000 psi, in some cases no more than 3000 psi, and in some cases even less (e.g., no more than 2000 psi). Various other pressure capacities may be used in other embodiments. Also, in some embodiments, pressure applied by the press may be progressively increased (e.g., a fraction of pressing time at a fraction of a maximum pressure to be applied, such as 15 seconds at ⅓ of the maximum pressure to be applied, followed by another fraction of the pressing time at another fraction of the maximum pressure to be applied, such as another 15 seconds at ⅔ of the maximum pressure to be applied, followed by yet another fraction of pressing time at the maximum pressure to be applied, such as 60 seconds at the maximum pressure to be applied). Hand shaping prior to placement into the press (e.g., into a mold of the press) may allow even lower pressure to be used by the press.

Finally, a solid or semi-solid amount of hashish product from step 150 and optionally step 160 can be cut at optional step 170 according to a pre-determined cutting pattern, a pre-determined weight, or a pre-determined length to obtain smaller units of hashish product for a pre-determined packaging size.

Advantageously, the pre-treated isolated cannabis trichomes do not need to undergo the mixing step 130 on the same day that the cannabis trichomes are pre-heated at step 220. In some examples, the pre-treated isolated cannabis trichomes could be stored for a period of up to a plurality of days (e.g., at least six days, or at least ten days, or at least 14 days), or up to 48 hours, or up to 24 hours, or up to 12 hours before being subjected to the mixing step 130 without significantly deteriorating the physical attributes (for example in terms of malleability, pliability, and/or crumbliness) of the hashish product. This facilitates the operation and logistics of the hashish production process 100 as there is less risk of producing degraded hashish products in cases where the pre-treated isolated cannabis trichomes cannot be mixed at step 130 on the same day as the pre-heating step 220.

Without being bound by any theory, it is believed that the herein described pre-treatment causes cannabis oils and/or resin to ooze out from the cannabis trichomes at least partially, which then form an oil layer on at least a surface thereof. This oil layer can be qualitatively observed upon performing the herein described pre-treatment step as the resulting pre-treated isolated trichomes have a “dark” appearance thereafter. It is believed that this oil layer may facilitate the adhesion of the isolated trichomes one to another during the subsequent mixing step without requiring the need for additional heating and/or pressing steps on the formed hashish product, as is typically performed in batch-like pressing processes of the prior art. It is believed that causing the proper balance of oil amounts oozing out from the cannabis trichomes is key to ultimately obtaining the desired hashish properties. The present inventors have herein described monitoring the decarboxylation level as one manner of monitoring and correlating the extent of pre-treatment to the desired oil amounts oozing out from the cannabis trichomes.

In some embodiments, the hashish product of the present disclosure is characterized as having a desirable dark color, such as having a lightness value L*≤50 on CIELAB scale.

The person of skill will readily understand that assessing and/or measuring the color can be performed quantitatively using a colorimeter, a spectrophotometer, or qualitatively with the human eye. For example, for quantitative assessment/measurement, the color can be measured by reflectance spectrophotometer ASTM standard test methodology. Tristimulus L*, a*, b* values are measured from the viewing surface of the hashish product. These L*, a*, b* values are reported in terms of the CIE 1976 color coordinate standard (CIELAB scale). L* is lightness which is the relative brightness of a surface with a range from 0-100, wherein L*=0 translates as darkest black and L*=100 translates as lightest white.

In some embodiments, the hashish product of the present disclosure has a lightness value L*≤50 based on the CIELAB scale—e.g., the reader will readily recognize that such lightness value range leaves flexibility to the producer, as the product can be made darker through other means if desirable to favor consumer appeal.

For example, in some embodiments, the hashish product may have a lightness value L* from 0 to about 50 or any value therebetween, or in a range of values defined by any values therebetween. For example, the hashish product may have a lightness value L* up to about 50, up to about 45, up to about 40, up to about 35, up to about 30, up to about 25, up to about 20, up to about 15, up to about 10, up to about 5 or any value therebetween. For instance, the hashish product may have a lightness value L* of from about 5 to about 45, of from about 10 to about 40, from about 15 to about 35, from about 20 to about 30. For example, the hashish product may have a lightness value L* of about 5, of about 10, of about 15, of about 20, of about 25, of about 30, of about 35, of about 40, of about 45, or about 50. For instance, in some embodiments, the lightness value L* may be no more than about 40, in some cases no more than about 30, in some cases no more than about 20, and in some cases no more than about 10.

Although trichome pre-treating (e.g., pre-heating) can afford many benefits, in some embodiments where a crude cannabis extract is incorporated with the isolated cannabis trichomes to be part of the hashish product, such trichome pre-treating may be omitted, i.e., dispensed with so that no pre-heating or other pre-treatment of cannabis trichomes is performed in making the hashish product. An example of such a variant of the process 100, denoted 100′, is shown in FIG. 1F where the pre-heating step 110 is omitted and replaced by step 112 in which the crude cannabis extract is incorporated with the isolated cannabis trichomes. For example, in some embodiments, the crude cannabis extract may be incorporated with the isolated cannabis trichomes in a relatively large proportion, as this may result in the hashish product having desired darkness and mechanical properties (e.g., malleability, etc.). For instance, in some embodiments, the content of the crude cannabis extract in the combination of the crude cannabis extract and the isolated cannabis trichomes may be at least 15 wt. %, in some cases at least 18 wt. %, and in some cases at least 20 wt. %. In some cases, by having the content of the crude cannabis extract in the combination of the crude cannabis extract and the isolated cannabis trichomes at 20 wt. %, the hashish product may be very dark (e.g., black) and malleable.

### Practical Implementation

There are several options to implement the herein described process 100.

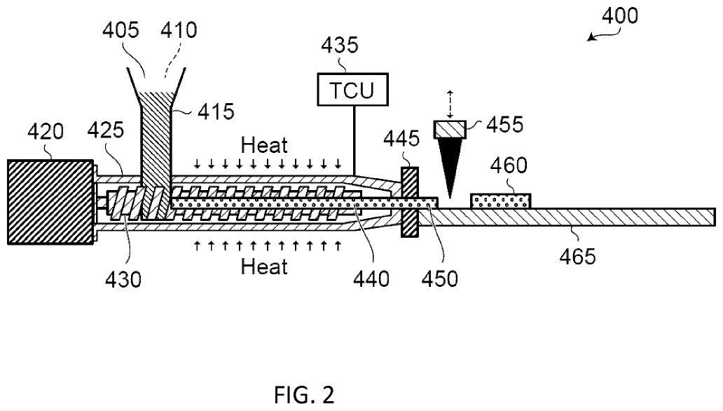

FIG. 2 illustrates a system 400 for implementing the process 100 to make individual units of hashish product 460 in accordance with an embodiment. The system 400 includes an extruder apparatus 425 that uses mechanical mixing means to amalgamate the pre-treated isolated cannabis trichomes 405 (and optionally one or more additional component(s) 410) into a coherent and substantially homogenous cohesive mass 450.

In this embodiment, the system 400 further comprises a feed hopper 415 through which the pre-treated isolated cannabis trichomes 405 (and optionally the one or more additional component(s) 410) are fed. As discussed previously, non-limiting examples of such one or more additional component(s) 410 include terpenes, flavonoids, water in the form of steam, ice or liquid, cannabinoids in the form of crude extracts, distillates, isolates, winterized cannabis extracts, rosin, shatter, or resins, or any combinations thereof. In another embodiment, at least a portion of the one or more additional component(s) 410 may be fed into the extruder apparatus 425.

The extruder apparatus 425 is powered by a motor 420 that drives at least one extruder screw 430 to apply pressure and mechanical shear on the pre-treated isolated cannabis trichomes 405 (and optionally the one or more additional component(s) 410) entering the extruder 425. For example, the extruder screw 430 may be configured for applying compression and shear forces to the pre-treated isolated cannabis trichomes 405 via a plurality of interpenetrate helicoidal surfaces present along at least a portion of the extruder screw 430.

When desired, the system 400 may also implement heating, such as within one or more predetermined portions (each a “heating zone”) of the extruder apparatus 425, or throughout the length of the extruder apparatus 425, depending on specifics applications. The operating parameters of the extruder apparatus 425, such as those discussed previously (e.g., the heating temperature and extruder screw rpm), can be selected to alter residence time of the resinous mixture 440 (or pre-treated isolated cannabis trichomes 405) in the extruder apparatus 425 to obtain the cohesive mass 450. Advantageously, it has been observed that operating parameters such as heat and extrusion speed change the pressure experienced at the die and may alter the characteristics of the hash product discussed above.

In some embodiments, the heating may additionally advantageously assist in homogeneous mixing of the pre-treated isolated cannabis trichomes 405 and optional additional components 410 to form the cohesive mass 450.

In some embodiments, the heating time may be of about 5 minutes, about 10 minutes, about 15 minutes, about 20 minutes, about 30 minutes, about 40 minutes, about 50 minutes, or about 60 minutes, depending on the specifics of an application, in each of the one or more heating zones of the extruder apparatus 425.

In some embodiments, the pressure applied by the extruder screw 430 is accompanied by heat to enhance mixing of the batch of pre-treated isolated cannabis trichomes 405 (and optionally the one or more additional component(s) 410), and/or further extract the resinous content of the pre-treated isolated cannabis trichomes and obtain a heated, cohesive, continuous and substantially homogenous resinous mixture 440.

In some embodiments, the heat may be applied through a heating element (not shown) that is embedded with the extruder screw 430 and extends along the entire or part(s) of the length of the extruder screw 430. In another embodiment, the heat may be applied through a heated jacket (not shown) that partially, or entirely, surrounds the extruder apparatus 425. To control the amount of heat input to the extruder and ensure that the quality of the resinous mixture 440 would not be compromised, a temperature controlling unit (TCU) 435 can also be associated with the extruder apparatus 425 to monitor heat within the extruder apparatus 425 and take any necessary action in the event of major deviations from the intended extrusion temperature.

For example, the temperature controlling unit (TCU) 435 may include a thermometer (not shown) that is connected to the exterior body of the extruder with its distal end in contact with the resinous mixture 440 recording an average resinous mixture temperature (T1). In another embodiment, the thermometer may be connected to the exterior body of the extruder apparatus 425 with its distal end attached to the outer surface of the extruder apparatus 425 recording an average operating temperature (T2) wherein T2=T1-AT with AT being a temperature offset.

The resinous mixture 440 then exits the extruder apparatus in the form of an elongated, continuous solid or semi-solid cohesive mass 450. Optionally, the extrusion apparatus 425 may include a die 445 at the outlet thereof, which may impart any pre-determined shape to the cohesive mass 450. At that point in the process, the long and continuous solid or semi-solid cohesive mass 450 can be subjected to ambient temperature and pressure.

A cutting means 455 may be placed downstream of the extruder die 445. The cutting means 455 may be configured to cut the cohesive mass 450 according to a pre-established cutting pattern. In a non-limiting example of implementation, the pre-established cutting pattern may comprise cutting the cohesive mass 450 along a transverse axis and at pre-determined time intervals to obtain hashish product unit 460 of a pre-determined length and/or weight. For example, to obtain a plurality of hashish product units 460 with consistent dimensions and/or weight, the cutting means 455 can act intermittently to cut the cohesive mass 450 into individual units of hashish product 460. The individual units of hashish product 460 could be further transferred onto a flat conveyor belt 465 or fall under gravity over an inclined conveyor belt (not shown) and sent for packaging and/or storage.

The system 400 may similarly be used in embodiments without trichome pre-treating (e.g., pre-heating) in which a crude cannabis extract may be incorporated with the isolated cannabis trichomes in a relatively large proportion, as discussed above.

### Density Determination

Density of solid/semi-solid materials such as a hashish product can be determined according to different methods and via different apparatus and according to different standards known in the art.

For solid/semi-solid materials with regular shapes and/or neat geometries, density can be determined by dividing the mass of the solid/semi-solid material by its volume. As a person skilled in the art would appreciate, for solid/semi-solid materials with irregular shapes and/or uneven surfaces, indirect methods may be used. In some cases, for example, the mass of the product sample can be separately determined with a balance while sample volume is determined by immersing the sample in a liquid container to determine volume of the product based on the volume of liquid that is displaced upon immersing the sample. In some other cases, for example, density can be calculated according to ASTM D1505 using a density column filled with liquid while reference balls with known densities are floated in the liquid column and the sample density can be determined based on its floating position in the density column and relative to the reference balls. In some other case, density can be calculated according to ASTM D792 using a density determination kit equipped with a balance to determine the mass of the sample both in the air and immersed conditions wherein the sample is immersed in a liquid of known density. Such density determination devices are based on hydrostatic weighting and Archimedean principle and are known to a person skilled in the art. Different models may be available including but not limited to Sartorius AG Density Determination Kit (models YDK03MS and YDK04MS), Mettler Toledo Density Determination Devices (available in the USA), and the like.

In one preferred embodiment, density determination is performed using a Sartorius Density Determination Kit (model YDK03MS or YDK04MS).

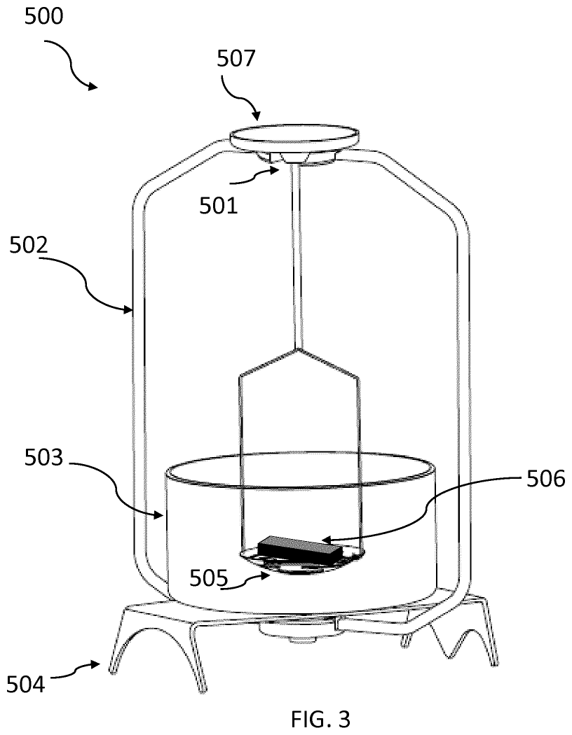

FIG. 3 a non-limiting example of a Sartorius device 500 employed to determine density of a sample of hashish product 506. The device for measuring density comprises a sample holder 505 fixed onto a hanger assembly 501 which is in turn supported by a bar frame 502. The sample holder 505 holding the hashish product sample 506 is immersed in a beaker 503 filled with a liquid (typically water—not shown). A thermometer may also be placed inside the beaker (not shown) to measure liquid temperature inside the beaker. The beaker 503 is placed on a metal support plate 504. The density of the hashish product sample 506 is then determined.

The “Density Test” consists of the following: weight of the hashish product sample 506 is determined both in air and in the liquid (immersed condition) using the hydrostatic balance 507, and then, the hashish product density is calculated according to the following formula:

$$density = \frac{W a * d w}{W a - W w}$$

wherein
Wa=weight of the hashish product sample in air,
dw=density of water at 21° C.,
ww=weight of the hashish product sample in water at 21° C.

### Three-Point Bend Test

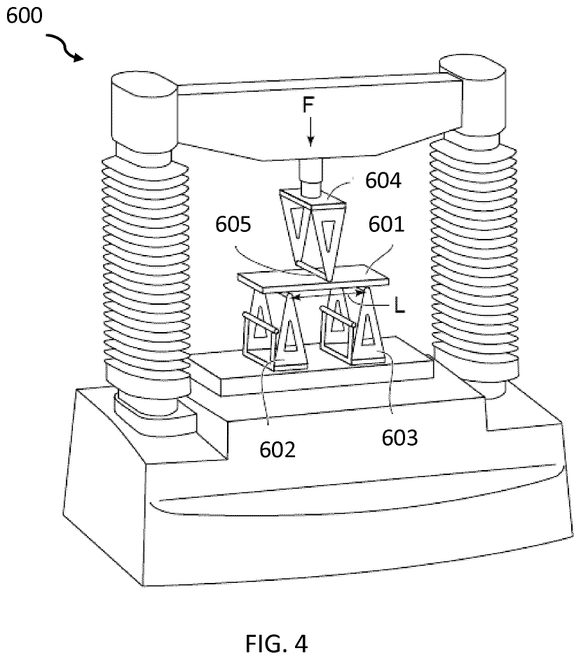

FIG. 4 is a non-limiting example of a three-point bend test employed to determine physical properties of the hash product. During the test, the force applied to and the displacement of the probe are recorded. A force-over displacement graph is typically generated and usually begins with a linear section that corresponds to elastic (reversible) deformation, then most samples show a curved section that shows plastic (irreversible) deformation. Different samples will give different load-distance responses—stronger and stiffer samples show higher forces, brittle samples break before any plastic deformation occurs and tough samples show a large area under the curve corresponding to a large amount of energy required for deformation.

The test procedure using a Texture Analyzing device 600 is as follows:
a. a 20 mm by 20 mm sample of hashish product 601 having a height of about 5-6 mm was placed on two support anvils 602/603 of the Texture Analyzing device 600, which anvils were distanced by a predetermined length (L),
b. a gradually descending probe 604 attached to a 100 kg load cell was landed on the center point 605 of the sample 601 while exerting a controlled vertical force F on the sample 601 until the sample 601 started to bend (elastic deformation) followed by a plastic deformation and eventually broke apart. A force-over-displacement graph was generated by the Texture analyzer software.

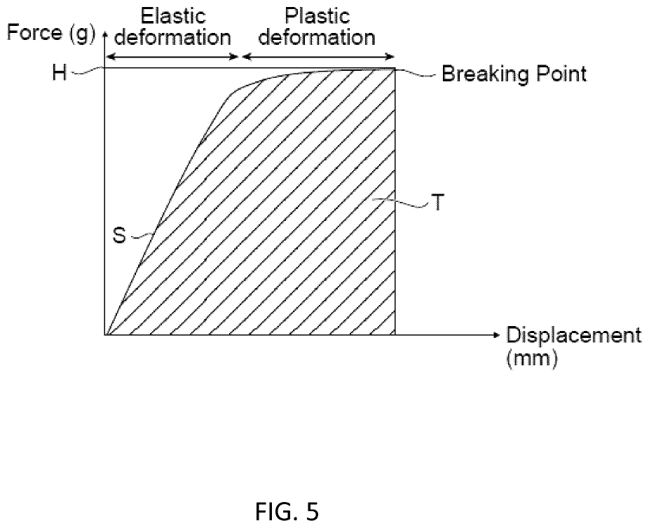

A non-limiting example of a force-over-displacement graph obtained from the three-point bend test is shown in FIG. 5, where the applied force F (expressed in grams) is plotted against the probe displacement (expressed in mm). The resulting graph includes a linear elastic deformation zone and a plastic deformation zone. The slope of the curve in the linear elastic deformation zone is equivalent to Stiffness (as shown by “S” in FIG. 5). The maximum force beyond which the sample breaks (breaking point) is equivalent to Hardness (as shown by “H” in FIG. 5). The area under the curve (expressed as gram*mm) is equivalent to Toughness (as shown by “T” in FIG. 5).

The three-point bend test can be performed with a Texture analyzer, such as the TA.XT Plus or TA.XT2 available from Stable MicroSystems (Surrey, United Kingdom), the TA-XT2i/5 texture analyzer from Texture Technologies Corp. (Scarsdale, N.Y), or any other texture analyzing instrument known to a person of skill in the art.

### Puncture Test

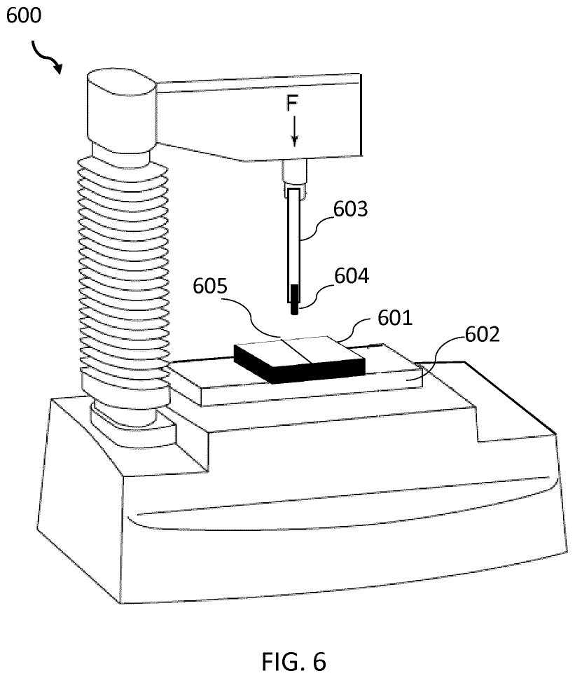

FIG. 6. is a non-limiting example of a puncture test employed to determine physical properties of the hashish product.

During the test, a force applied to a sample by, and a displacement of, a probe are recorded. A force-over displacement graph is typically generated and usually begins with an ascending linear section that corresponds to elastic (reversible) deformation which reaches to a maximum peak as sign of sample puncture, then samples show a sharp descending section that shows plastic (irreversible) deformation post-puncture. Different samples will give different load-distance responses—stronger and harder samples show higher forces, softer samples puncture faster. Tough samples show a large area under the curve corresponding to a large amount of energy required for deformation (puncture).

The test procedure using a Texture Analyzing device 600 is as follows:
a. a 20 mm by 20 mm sample of hashish product 601 having a height of about 5-6 mm was placed on a flat surface 602 of the Texture Analyzing device 600,
b. a gradually descending probe 603 (comprising a 2 mm tip 604) attached to a 100 kg load cell was landed on the center point 605 of the sample 601 while exerting a controlled vertical force F on the sample 601 until the sample 601 is punctured. A force-over-displacement graph was generated by the Texture analyzer software.

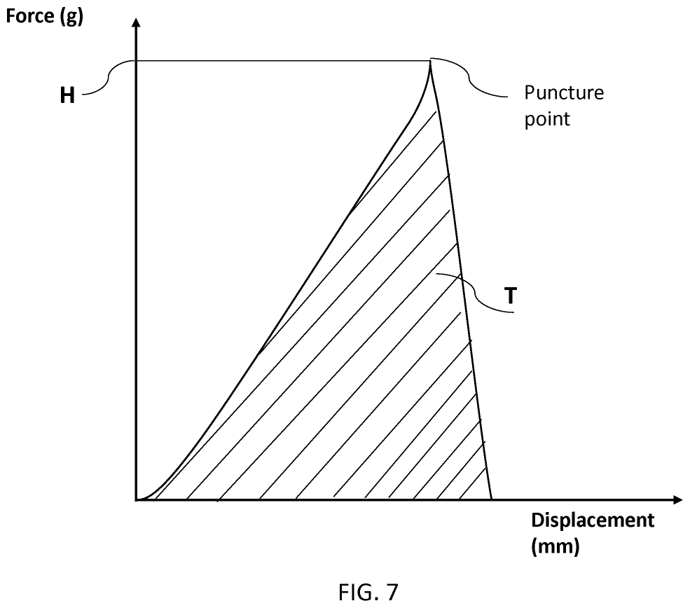

A non-limiting example of a force-over-displacement graph obtained from the puncture test is shown in FIG. 7, where the applied force F (expressed in grams, “g”) is plotted against the probe displacement (expressed in mm). The resulting graph includes an ascending linear elastic deformation zone reaching a peak followed by a sharp descending zone. The maximum force beyond which the sample punctures (puncture point) is equivalent to Hardness (as shown by “H” in FIG. 7). The area under the curve (expressed as g*mm) is equivalent to Toughness (as shown by “T” in FIG. 7).

The puncture test can be performed with a Texture analyzer, such as the TA.XT Plus or TA.XT2 available from Stable MicroSystems (Surrey, United Kingdom), or any other texture analyzing instrument known to a person of skill in the art.

### EXAMPLES

The following examples are for illustrative purposes only and are not meant to limit the scope of the compositions and methods described herein. These examples are for illustrative purposes only and are not meant to limit the scope of the compositions and methods described herein.

### Example 1: Pre-Heating Isolated cannabis Trichomes at 120° C.

In this example, a batch (Kief ID BBI-088) of isolated cannabis trichomes (NLxBB cannabis strain) was placed in an oven at 120° C. for pre-heating same. Samples were retrieved from the oven pre-determined pre-heating time duration (namely at 20 min, 40 min, 60 min, and 80 min). The decarboxylation level of the samples was measured with high performance liquid chromatography (HPLC) using THC-A and THC content. The results are summarized in Table 1.

TABLE 1

| | Pre-heating | | | Total | |
|---|---|---|---|---|---|
| Sample | duration | [THC] | [THC-A] | [THC] | Ratio |
| # | (min) | (wt. %) | (wt. %) | (wt. %) | THC:THC-A |
| 1 | 0 | 0.30 | 33.7 | 32.60 | 1:100 |
| 2 | 20 | 21.34 | 13.56 | 33.23 | 1.5:1 |
| 3 | 40 | 32.87 | 0.69 | 33.48 | 50:1 |
| 4 | 60 | 33.81 | 0.28 | 34.05 | 100:1 |
| 5 | 80 | 33.46 | 0.17 | 33.61 | 150:1 |

Table 1 shows that pre-heating isolated NLxBB cannabis trichomes at 120° C. for 20 min affords partly decarboxylated isolated cannabis trichomes. In contrast, pre-heating at 120° C. for 40 min was sufficient to obtain substantially complete decarboxylation.

### Example 2: Pre-Heating at 120° C.

In this example, a batch (Kief ID RND0004150-01) of isolated cannabis trichomes (SL cannabis strain) was placed in an oven at 120° C. for pre-heating same. Samples were retrieved from the oven pre-determined pre-heating time duration (namely at 5 min, 10 min, 15 min, 20 min, 25 min, and 30 min). The decarboxylation level of the samples was measured with HPLC using THC-A and THC content. The results are summarized in Table 2.

TABLE 2

| | Pre-heating | | | Total | |
|---|---|---|---|---|---|
| Sample | duration | [THC] | [THC-A] | [THC] | Ratio |
| # | (min) | (wt. %) | (wt. %) | (wt. %) | THC:THC-A |
| 1 | 0 | 5.5 | 27.52 | 29.64 | 1:5 |
| 2 | 5 | 5.79 | 28.69 | 30.95 | 1:5 |
| 3 | 10 | 5.84 | 26.85 | 29.38 | 1:5 |
| 4 | 15 | 6.47 | 27.34 | 30.45 | 1:4 |
| 5 | 20 | 7.59 | 26.9 | 31.18 | 1:3 |
| 6 | 25 | 22.77 | 7.68 | 29.5 | 3:1 |
| 7 | 30 | 25.7 | 5.1 | 30.17 | 5:1 |

Table 2 shows that pre-heating isolated SL cannabis trichomes at 120° C. from 15 min (sample #4) up to 30 min (sample #7) results in partial decarboxylation.

### Example 3: Pre-Heating at 100° C.

In this example, a batch (Kief ID BBI-087) of isolated cannabis trichomes (NLxBB cannabis strain) was placed in an oven at 100° C. for pre-heating same. Samples were retrieved from the oven pre-determined pre-heating time duration (namely 20 min, 30 min, 40 min, 50 min, 60 min, 70 min and 80 min). The decarboxylation level of the samples was measured with HPLC using THC-A and THC content. The results are summarized in Table 3.

TABLE 3

| | Pre- | | | Total | |
|---|---|---|---|---|---|
| Sample | heating | [THC] | [THC-A] | [THC] | Ratio |
| # | duration | (wt. %) | (wt. %) | (wt. %) | THC:THC-A |
| 1 | 20 | 5.12 | 27.37 | 29.13 | 1:5 |
| 2 | 30 | 7.87 | 24.91 | 29.71 | 1:3 |
| 3 | 40 | 8.78 | 22.85 | 28.82 | 1:2 |
| 4 | 50 | 10.12 | 20.21 | 27.84 | 1:2 |
| 5 | 60 | 14.11 | 16.20 | 28.32 | 1:1 |
| 6 | 70 | 15.60 | 13.87 | 27.76 | 1:1 |
| 7 | 80 | 19.57 | 9.10 | 27.55 | 2:1 |

Table 3 shows that pre-heating isolated NLxBB cannabis trichomes at 100° C. from 20 min (sample #1) up to 80 min (sample #7) results in partial decarboxylation.

### Example 4: Pre-Heating at 80° C.

In this example a batch (Kief ID BBI-087) of isolated cannabis trichomes (NLxBB cannabis strain) was placed in an oven at 80° C. for pre-heating same. Samples were retrieved from the oven pre-determined pre-heating time duration (namely 20 min, 30 min, 40 min, 50 min, 60 min, 70 min, 80 min, 90 min and 120 min). The decarboxylation level of the samples was measured with HPLC using THC-A and THC content. The results are summarized in Table 4.

TABLE 4

| | Heating | | | Total | |
|---|---|---|---|---|---|
| Sample | duration | [THC] | [THC-A] | [THC] | Ratio |
| # | (min) | (wt. %) | (wt. %) | (wt. %) | THC:THC-A |
| 1 | 20 | 3.12 | 29.37 | 28.88 | 1:9 |
| 2 | 30 | 3.28 | 31.21 | 30.65 | 1:10 |
| 3 | 40 | 3.39 | 28.21 | 28.14 | 1:9 |
| 4 | 50 | 4.00 | 29.61 | 29.97 | 1:7 |
| 5 | 60 | 4.14 | 28.89 | 29.48 | 1:7 |
| 6 | 70 | 4.11 | 28.08 | 28.73 | 1:7 |
| 7 | 80 | 4.23 | 27.73 | 28.56 | 1:6 |
| 8 | 90 | 4.69 | 26.92 | 28.30 | 1:6 |
| 9 | 120 | 5.12 | 26.38 | 28.26 | 1:5 |

Table 4 shows that pre-heating isolated NLxBB cannabis trichomes for 50 min at 80° C. (sample #4) initiates partial decarboxylation. Partial decarboxylation is still obtained with pre-heating time up to 120 min (sample #9).

### Example 5: Extruding Pre-Treated Isolated Cannabis Trichomes

In this example, a batch (BBE-036) of isolated cannabis trichomes (NLxBB cannabis strain) was placed in an oven at 120° C. for 25 minutes for pre-heating same. The decarboxylation level was measured with high performance liquid chromatography (HPLC) using THC-A and THC content. The results are summarized in Table 5.

TABLE 5

| heating | | | Total | |
|---|---|---|---|---|
| duration | [THC] | [THC-A] | [THC] | Ratio |
| (min) | (wt. %) | (wt. %) | (wt. %) | THC:THC-A |
| 25 | 16.01 | 14.43 | 28.67 | 1:1 |

The water content of the pre-treated isolated trichomes was adjusted to 10% and hashish products were then made by mixing the pre-treated isolated cannabis trichomes samples in an extruder apparatus (hereinafter, “test hashish”). Control hashish products were also made by mixing non-treated isolated cannabis trichomes (hereinafter, “control hashish”).

A batch of 150 g of the pre-treated isolated cannabis trichomes was mixed thoroughly and placed into the hopper of an ETPI Lab extruder (The Bonnot Company, USA). The extruder was operated with the following extrusion operating parameters: temperature of 60° C. and screw speed of 15 rpm. A cohesive mass was retrieved through an extrusion die (20 mm×5 mm) of the extruder and cut to obtain a 20 mm long test hashish product. The same procedure was followed with non-treated isolated cannabis trichomes to obtain a similar sized control hashish.

The resulting control hashish was dark in color hard and easily broke with hand pressure. The resulting test hashish was dark in color hard and had acceptable malleability and was slightly tough and bendy.

### Example 6: Physical Characterization of the Hashish Products from Example 5

Physical properties of the control and test hashish products from Example 5 were determined with dimension measurements and texture assessment.

The dimension measurements [(height (H), width (W) and length (L)] are summarized in Tables 6A-6B.

TABLE 6A
Test hashish

| | | | | Unit |
|---|---|---|---|---|
| | | | | volume |
| Product # | H (mm) | W (mm) | L (mm) | (mm³) |
| 1 | 6.08 | 21.10 | 21.65 | 2777.44 |
| 2 | 6.02 | 21.10 | 21.57 | 2739.86 |
| 3 | 6.12 | 21.06 | 20.88 | 2691.16 |
| 4 | 6.14 | 21.22 | 22.36 | 2913.30 |
| 5 | 6.06 | 21.11 | 20.99 | 2685.18 |
| 6 | 6.15 | 21.32 | 20.45 | 2681.36 |
| 7 | 6.18 | 21.27 | 21.96 | 2886.61 |
| 8 | 6.11 | 21.18 | 21.15 | 2737.02 |
| 9 | 6.16 | 21.23 | 20.42 | 2670.46 |
| 10 | 6.12 | 21.01 | 21.07 | 2709.21 |
| 11 | 6.18 | 21.18 | 20.67 | 2705.55 |
| 12 | 6.17 | 21.26 | 21.48 | 2817.62 |
| Average | 6.12 | 21.17 | 21.22 | 2751.23 |
| Standard | 0.05 | 0.09 | 0.60 | 81.60 |
| deviation | | | | |
| Coefficient | 0.01 | 0.00 | 0.03 | 0.03 |
| of variance | | | | |

TABLE 6B
control hashish

| | | | | Unit |
|---|---|---|---|---|
| | | | | volume |
| Product # | H (mm) | W (mm) | L (mm) | (mm³) |
| 1 | 5.41 | 20.36 | 20.00 | 2202.95 |
| 2 | 5.38 | 20.32 | 20.00 | 2186.43 |
| 3 | 5.49 | 20.42 | 20.00 | 2242.12 |
| 4 | 5.41 | 20.35 | 20.00 | 2201.87 |
| 5 | 5.47 | 20.37 | 20.00 | 2228.48 |
| 6 | 5.45 | 20.31 | 20.00 | 2213.79 |
| Average | 5.44 | 20.36 | 20.00 | 2212.61 |
| Standard | 0.04 | 0.04 | 0.00 | 18.34 |
| deviation | | | | |
| Coefficient | 0.01 | 0.00 | 0.00 | 0.01 |
| of variance | | | | |

The three-point bend test results are summarized in Table 7.

TABLE 7

| | | Three Point Bend Test | | |
|---|---|---|---|---|
| | | Stiffness | Hardness | Toughness |
| | | (g/mm) | (g) | (g*mm) |
| Control hashish | Average: | 7848 | 9278 | 11488 |
| | Standard | 702 | 366 | 1595 |
| | deviation: | | | |
| Test Hashish | Average: | 3003 | 4605 | 22095 |
| | Standard | 177 | 155 | 1169 |
| | deviation: | | | |

The results show that while both the control hashish and test hashish have similar color characteristics, they differ in terms of malleability properties—extruding pre-treated isolated trichomes affords a hashish product that has more user-desired malleable properties.

Moreover, Tables 6A-6B show that the test hashish product has a larger expansion ratio (cross-sectional, longitudinal, and volumetric) than the control hashish. Indeed, the test hashish witnessed an expansion from the initial die size imparted shape (5 mm×20 mm) and length (20 mm) to an expanded shape (average 6.12 mm×21.17 mm) and length (21.22 mm), thus resulting in a volumetric expansion from the initial volume of 2000 mm³ to an average volume of 2751 mm³. Conversely, any expansion for the control hashish was relatively negligible. The larger expansion ratio for the test hashish product is indicative that structural changes have occurred during and/or post extrusion which may be indicative of an increased internal porous structure.

Furthermore, Table 7 shows that the test hashish has desired malleability properties, namely in terms of stiffness, hardness and/or toughness.

### Example 7: Extruding Pre-Treated Isolated cannabis Trichomes with CBD Crude Input

In this example, a batch (mix of BBI-193-A and BBI-193-B) of isolated cannabis trichomes (Snow Leopard (SL)) was pre-heated under different operation conditions as follows and subjected to extrusion:

TABLE 8

| Operating | | | |
|---|---|---|---|
| condition | Input | Pre-heating | Extrusion |
| A | Isolated cannabis | Oven at 120° C. for 40 min (thin | Number of passes: 4 |
| | trichomes: 440 gr, | spreading of trichomes) | Die size: ¼ in. |
| | CBD crude input: 25 | | Mixing speed: low |
| | gr (5% wt.), water: 0% | | Mixing adaptor: whisk |
| | | | type |
| B | Isolated cannabis | Oven at 120° C. for 60 min (thin | Number of passes: 2.5 |
| | trichomes: 440 gr, | spreading of trichomes) | Die size: ¼ in. |
| | CBD crude input: 25 | | Mixing speed: low |
| | gr (5% wt.), water: 0% | | Mixing adaptor: whisk |
| | | | type |
| C | Isolated cannabis | Oven at 120° C. for 40 min (thin | Number of passes: 2 |
| | trichomes: 440 gr, | spreading of trichomes) | Die size: ¼ in. |
| | CBD crude input: 50 | | Mixing speed: low |
| | gr (10% wt.), water: 0% | | Mixing adaptor: whisk |
| | | | type |
| D | Isolated cannabis | Oven at 120° C. for 60 min (thin | Number of passes: 2 |
| | trichomes: 440 gr, | spreading of trichomes) | Die size: ¼ in. |
| | CBD crude input: 50 | | Mixing speed: low |
| | gr (10% wt.), water: 0% | | Mixing adaptor: whisk |
| | | | type |

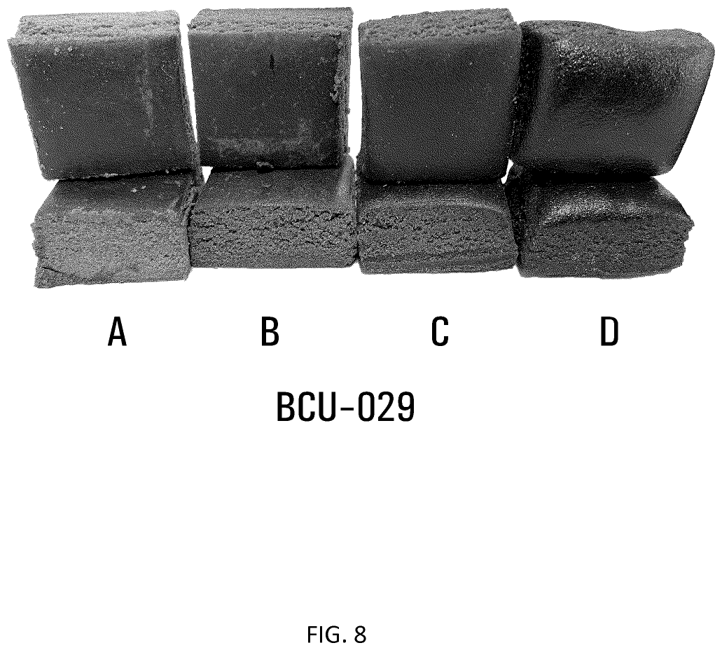

The hashish products obtained under operating conditions A, B, C and 0 are shown in FIG. 8 (test code: BCU-029) all appearing as cohesive masses that were dark in color. It was found that pre-heating the isolated cannabis trichomes for 60 minutes reduced the number of extrusion passes from 4 passes (operating condition A) to 2.5 passes (operating condition B) when only 5% wt. of CEO crude oil is input to the extruder. However, at 10% wt. of CEO crude oil input, the number of passes does not change when increasing the pre-heating time from 40 minutes (operating condition C) to 60 minutes (operating condition D). The difference between the hashish products corresponding to operating conditions C and D is that the hashish product of operating condition D is darker in appearance. More crude input combined with longer heating may thus promote darker and more malleable products.

The hashish products obtained under operating conditions A, B, C and 0 were also subjected to three-point bend test as well as puncture test to determine their physical characteristics as shown in Table 9.

TABLE 9

| | Three-point Bend Test | | | | | |
|---|---|---|---|---|---|---|
| | | | | Sample | Puncture Test | |
| Hashish | Stiffness | Hardness | Toughness | height | Hardness | Toughness |
| product | (g/mm) | (g) | (g · mm) | (mm) | (g) | (g · sec) |
| Operating | 2548.386 ± | 6058.245 ± | 23349.315 ± | 10.628 ± | 2298.984 ± | 2176.579 ± |
| condition A | 351.594 | 402.232 | 3505.924 | 0.21 | 99.356 | 245.957 |
| Operating | 819.306 ± | 4155.567 ± | 30169.240 ± | 10.830 ± | 1237.976 ± | 1157.869 ± |
| condition B | 136.122 | 339.107 | 3558.347 | 0.149 | 80.508 | 43.669 |
| Operating | 300.736 ± | 2044.218 ± | 15705.133 ± | 10.429 ± | 725.753 ± | 561.778 ± |
| condition C | 16.423 | 167.335 | 1110.142 | 0.558 | 64.215 | 34.111 |
| Operating | 153.108 ± | 1383.136 ± | 14466.772 ± | 10.528 ± | 413.579 ± | 318.078 ± |
| condition D | 28.181 | 350.196 | 3466.302 | 0.204 | 62.111 | 32.361 |

### Example 8: Extruding Re-Treated Isolated cannabis Trichomes with CBD Crude Input

In this example, and based on results from Example 7 wherein the effect of pre-heating time and amount of CEO input was assessed, a batch (PR06627-KIEF-01) of isolated cannabis trichomes (Snow Leopard (SL)) was pre-heated under different operation conditions as follows and subjected to extrusion:

TABLE 10

| Operating | | | |
|---|---|---|---|
| condition | Input | Pre-heating | Extrusion |
| E | Isolated cannabis | Oven at 120° C. for 60 min (thin | Number of passes/die |
| | trichomes: 420 gr, | spreading of trichomes) | size: 2 passes/¼ in. |
| | CBD crude input: 0 | | followed by 5 passes/ |
| | gr, water: 0% | | 5/32 in. |
| | | | Mixing speed: — |
| | | | Mixing adaptor: — |
| F | Isolated cannabis | Oven at 120° C. for 60 min (thin | Number of passes/die |
| | trichomes: 420 gr, | spreading of trichomes) | size: 4 passes/¼ in. |
| | CBD crude input: 24 | | Mixing speed: low |
| | gr (5% wt.), water: 0% | | Mixing adaptor: whisk |
| | | | type |
| G | Isolated cannabis | Oven at 120° C. for 40 min (thin | Number of passes/die |
| | trichomes: 420 gr, | spreading of trichomes) | size: 4 passes/¼ in. |
| | CBD crude input: 47 | | Mixing speed: low |
| | gr (10% wt.), water: 0% | | Mixing adaptor: whisk |
| | | | type |

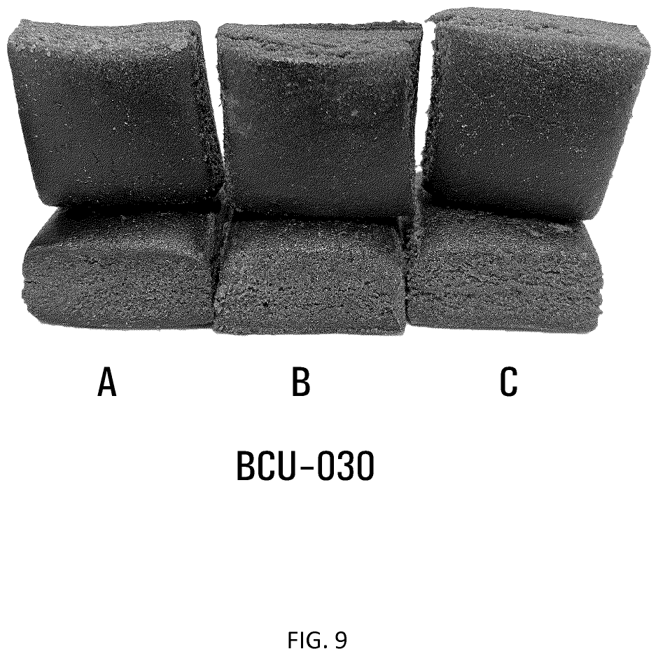

The hashish products obtained under operating conditions E, F and G are shown in FIG. 9 (test code: BCU-030) all appearing as cohesive masses that were dark in color. Regarding operating condition E, it was observed that in the absence of crude CBD oil and despite pre-heating the isolated cannabis trichomes for 60 minutes at 120° C., it is possible to use a combination of a ¼ in. die size followed by a smaller die size of 5/32 in. in order to increase the shear action during extrusion and obtain a product with desired color and malleability (it was further observed that if a sole 5/32 in. die size is used, the isolated cannabis trichomes may not be able to pass through the die). This method was found to be useful for obtaining hashish products with desired color and malleability if no CBD crude oil is used.

As for the operating conditions F and G, wherein 5% wt. and 10% wt. of CBD crude oil were used respectively, it was observed that a single ¼ in die would pass the isolated cannabis trichomes. It was further observed that the hashish products started to get a black color after 2 passes of extrusion while achieving the desired appearance after 4 passes.

The hashish products obtained under operating conditions E, F and G were also subjected to three-point bend test and puncture test to determine their physical characteristics as shown in Table 11.

TABLE 11

| | Three-point Bend Test | | | | | |
|---|---|---|---|---|---|---|
| | | | | Sample | Puncture Test | |
| Hashish | Stiffness | Hardness | Toughness | height | Hardness | Toughness |
| product | (g/mm) | (g) | (g · mm) | (mm) | (g) | (g · sec) |
| Operating | 199.221 ± | 1822.300 ± | 17691.448 ± | 9.254 ± | 531.525 ± | 401.825 ± |
| condition E | 38.926 | 229.581 | 2361.728 | 1.040 | 59.108 | 134.660 |
| Operating | 146.876 ± | 1527.506 ± | 14933.863 ± | 10.025 ± | 491.973 ± | 414.017 ± |
| condition F | 30.274 | 70.255 | 517.251 | 0.189 | 21.664 | 20.742 |
| Operating | 255.952 ± | 1963.245 ± | 21236.399 ± | 10.426 ± | 575.172 ± | 498.369 ± |
| condition G | 36.852 | 131.982 | 1664.079 | 0.482 | 18.342 | 47.665 |

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art, such as for example+/−20%, +/−15%, +/−10%, or +/−5%. Hence, numerical quantities given herein generally include such error margin such that the terms “around”, “about” or “approximately” can be inferred if not expressly stated.

As used throughout the present disclosure, the terms “concentration” and “content” are used interchangeably and refer to the weight or mass fraction of a constituent, i.e., the weight or mass of a constituent divided by the total mass of all constituents, and is expressed in wt. %, unless stated otherwise.

Although various embodiments of the disclosure have been described and illustrated, it will be apparent to those skilled in the art considering the present description that numerous modifications and variations can be made. The scope of the invention is defined more particularly in the appended claims.

1. A process of making a hashish product, comprising:
a) providing isolated cannabis trichomes pre-treated to comprise a cannabis oil layer on at least a portion of a surface thereof;
b) mixing the isolated cannabis trichomes under conditions sufficient to obtain a resinous mixture; and
c) retrieving at least a portion of the resinous mixture through an extrusion die to obtain the hashish product comprising a cohesive mass of the isolated cannabis trichomes.

2. The process according to claim 1, wherein the step a) comprises pre-heating the isolated cannabis trichomes under conditions to obtain at least partial decarboxylation of one or more cannabinoid(s) of the isolated cannabis trichomes.

3. The process according to claim 1, wherein the step a) comprises (i) pre-heating cannabis material under conditions to obtain at least partial decarboxylation of one or more cannabinoid(s) of the cannabis material and (ii) isolating cannabis trichomes therefrom to obtain the isolated cannabis trichomes.

4. The process according to claim 2, wherein the pre-heating is performed at a temperature of from about 70° C. to about 130° C., preferably from about 80° C. to about 120° C., more preferably about 120° C.

5. The process according to claim 2, wherein the pre-heating is performed for a duration of from about 10 minutes to about 80 minutes, preferably from about 40 minutes to about 60 minutes, even more preferably from about 50 minutes to about 60 minutes.

6. The process according to claim 2, wherein the pre-heating is performed to obtain a decarboxylation level of the one or more cannabinoid(s) of from about 30% to about 100%, preferably from about 40% to about 80%, more preferably from about 50% to about 70%, and even more preferably about 60%.

7. The process according to claim 2, wherein the pre-heating is performed to obtain a ratio of a decarboxylated to acid form content of the one or more cannabinoid(s), by weight, of from about 100:1 to about 1:100, preferably from about 1:4 to about 50:1, more preferably from about 1:3 to about 5:1, even more preferably from about 2:1 to about 4:1, and yet even more preferably about 3:1.

8. The process according to claim 2, wherein the pre-heating step is performed on a trichome-containing layer of at least 5 mm.

9. The process accordingly to claim 8, wherein the trichome-containing layer is no more than 10 mm.

10. The process according to claim 1, wherein the hashish product comprises a not negligible content in acid form of one or more cannabinoid(s).

11. The process according to claim 10, wherein the not negligible content in the acid form of the one or more cannabinoid(s) of the hashish product is of no less than 1 wt. %, preferably of no less than 3 wt. %, even more preferably of no less than 5 wt. %.

12. The process according to claim 10, wherein the not negligible content in the acid form of the one or more cannabinoid(s) of the hashish product is of up to about 30 wt. %, preferably up to about 25 wt. %, more preferably up to about 20 wt. %.

13. The process according to claim 10, wherein the acid form of the one or more cannabinoid(s) of the hashish product comprises tetrahydrocannabinolic acid (THC-A), cannabidiolic acid (CBD-A), or THC-A and CBD-A.

14. The process according to claim 2, wherein the pre-heating is performed to obtain a decarboxylation level of the one or more cannabinoid(s) of from about 50% to about 100%, preferably from about 70% to about 100%, more preferably from about 90% to about 100%, and even more preferably about 100%.

15. The process according to claim 1, further comprising incorporating water to the pre-treated isolated cannabis trichomes prior to step b) to have a water content of about 20 wt. % or less, preferably between about 5 wt. % and about 15 wt. %, more preferably from about 10 wt. % to about 15 wt. %.

16. The process according to claim 1, wherein the extrusion die is a first extrusion die; and the process comprises passing the resinous mixture through a second extrusion die smaller than the first extrusion die to obtain the hashish product comprising the cohesive mass of the isolated cannabis trichomes.

17. The process according to claim 1, further comprising cutting the hashish product according to a pre-established cutting operational parameter.

18. (canceled)

19. The process according to claim 1, further comprising incorporating one or more of one or more cannabinoid(s), one or more terpene (s), one or more flavonoid(s), water, one or more flavoring agent(s), one or more non-toxic coloring agent(s), or a mixture thereof.

20. (canceled)

21. The process according to claim 19, wherein the one or more cannabinoid(s) is in the form of a crude cannabis extract, a cannabis distillate, a cannabis isolate, a winterized cannabis plant extract, cannabis rosin, cannabis resin, cannabis wax, cannabis shatter, or any combination thereof.

22. (canceled)

23. (canceled)

24. The process according to claim 1, further comprising incorporating a crude cannabis extract with the isolated cannabis trichomes.

25-47. (canceled)
